(19) United States
(12) Patent Application Publication
(10) Pub. No.: US 2020/0071736 A1
(43) Pub. Date: Mar. 5, 2020

(54) METHOD FOR THE ENZYMATIC SACCHARIFICATION OF A POLYSACCHARIDE

(71) Applicant: THE ROYAL INSTITUTION FOR THE ADVANCEMENT OF LEARNING/MCGILL UNIVERSITY, Montreal (CA)

(72) Inventors: FABIEN HAMMERER, MONTREAL (CA); LEIGH-ANNE LOOTS, MONTREAL (CA); JEAN-LOUIS DO, BROSSARD (CA); CHRISTOPHER W. NICKELS, MONTREAL (CA); TOMISLAV FRISCIC, MONTREAL (CA); KARINE AUCLAIR, LAVAL (CA)

(21) Appl. No.: 16/490,282

(22) PCT Filed: Mar. 1, 2018
(86) PCT No.: PCT/CA2018/050237

§ 371 Date: Aug. 30, 2019

Related U.S. Application Data
(60) Provisional application No. 62/465,443, filed on Mar. 1, 2017.

(51) Int. Cl. C12P 19/02; C12N 9/42; C12N 9/24; C12P 19/14

(52) U.S. Cl. CPC C12P 19/02; C12N 9/2437; C12Y 302/01014; C12N 9/248; C12P 19/14; C12N 9/2442

(57) ABSTRACT

A method for the enzymatic saccharification of a polysaccharide is provided. This method comprises the step a) of contacting the polysaccharide with a hydrolase and water, in the absence of solvent, thereby forming a solid reaction mixture; and the step b) of: b)-i. mixing and then incubating the solid reaction mixture, b)-ii. milling the solid reaction mixture, or b)-iii. milling and then incubating the solid reaction mixture.

### CROSS REFERENCE TO RELATED APPLICATIONS

This application claims benefit, under 35 U.S.C. § 119(e), of U.S. provisional application Ser. No. 62/465,443, filed on Mar. 1^{st}, 2017. All documents above are incorporated herein in their entirety by reference.

### FIELD OF THE INVENTION

The present invention relates to method for the enzymatic saccharification of a polysaccharide. More specifically, the present invention is concerned with such a method where the enzymatic saccharification occurs in a solvent-free environment.

### BACKGROUND OF THE INVENTION

Large scale production of bioethanol has become a worldwide priority as fossil fuel reserves dwindle and become less profitable due to increasing extraction costs. Biofuels constitute a renewable source of energy which, if properly harnessed and regulated, could address the looming energy crisis. Mass production however remains problematic to this day since the main sources of bioethanol come from food stocks such as starch.

Recently, attention has been shifting towards cellulosic ethanol, namely ethanol resulting from cellulose hydrolysis, the most abundant biopolymer in nature. Many types of biomass, such as wood, agricultural waste, grassy crops and solid rural waste are suitable to produce ethanol. These materials consist basically of cellulose, hemicellulose, and lignin.

Cellulose is a water-insoluble linear polysaccharide composed of units of D-glucose. The production of ethanol from cellulose first requires the breakdown of cellulose into simpler water-soluble carbohydrates, such as glucose and oligosaccharides of cellulose (i.e. oligocelluloses). The chemical breakdown of a polysaccharide, such as cellulose, into simpler molecules is generally called saccharification. Typically, for this process, the cellulose is either dissolved or suspended in a liquid. Once the cellulose has been converted to fermentable sugars, e.g., glucose, the fermentable sugars are easily fermented by yeast into ethanol. The sugars can also be catalytically converted or fermented to other chemicals besides ethanol.

There are two principal catalysts for the saccharification process of cellulose: acids (most often sulfuric acid) and cellulolytic enzymes (also called cellulases).

A principal technique for hydrolytic breakdown of cellulose is based on acidic hydrolysis, typically in dilute sulfuric acid, leading to smaller oligomeric products, as well as nanocellulose particles. These are typically sulfonated. Oligomeric cellulose breakdown products can be further broken down into smaller components through chemical modification or enzyme-catalyzed processes All of these require the isolation of the cellulose breakdown products, and enzyme catalysis will not work in the initially acidic environment. Solid-state (solvent-free) breakdown of cellulose involving an acidic (or basic) solid catalysts have also been proposed.

Usually, treatment with cellulolytic enzymes typically requires pre-treatment of the cellulose and is conventionally performed by mixing the substrate (lignocellulose material) with water to obtain a suspension of the cellulose mass, and then adding the enzymes. Hydrolysis is typically conducted over several hours or even several days. Once hydrolysis is over, the desired products are in the liquid portion of the reaction mixture, while unhydrolyzed cellulose, lignin and other insoluble components of the substrate remain in the solid portion. The desired products are isolated by filtering the suspensions and washing the solid.

Regretfully, so far the method of treatment of the cellulose containing stock with enzymes have failed to produce glucose and other fermentable sugars sufficiently cheaply that would make the process of ethanol production profitable. Even applying the most effective methods of pre-treatment, the amount of enzymes needed to convert the polysaccharides in the lignocellulose stock into fermentable carbohydrates is too large. When a lesser amount of cellulolytic enzymes is used, the glucose yield drops and treatment is longer, which makes the process unprofitable. Several methods have been proposed to reduce the quantity of enzyme needed. One of them combines hydrolysis with yeast fermentation, but it is rather inefficient. The combination of saccharification and yeast fermentation is not particularly beneficial because the optimum temperature to activate the yeast is much lower than the optimum temperature of activation of the enzymes. When carried out at a moderate temperature, this method is ineffective and causes the development of vulgar microflora. In an effort to overcome these problems, various cellulose pre-treatments (i.e. treatments applied before the enzymatic saccharification) have been suggested.

On another subject, mechanochemistry (or mechanical chemistry) is a branch of chemistry concerned with chemical and physico-chemical changes of substances due to the influence of mechanical energy. Mechanochemistry couples mechanical and chemical phenomena. It uses mechanical action to cause, sustain or modify chemical and physico-chemical changes in a substance. For example, ball milling is a mechanochemical technique that can be used to impart mechanical force and/or mechanical agitation to a substance to achieve chemical processing and transformations.

The mechanisms of mechanochemical transformations are often complex and are often quite different from usual thermal or photochemical mechanisms. Indeed, mechanochemistry is radically different from the traditional way of dissolving, heating and stirring chemicals in a solution or dispersion. In fact, mechanochemistry is most often conducted in the absence of bulk solvent. Indeed, when a liquid is present, it is only used in very small amounts. Hence, mechanochemistry is quite different from wet chemistry, including chemistry of slurries and suspensions.

In fact, it has become clear that removing the solvent from reactions can change reaction pathways considerably. The absence of a solvent during a mechanochemical synthesis can have varied consequences including, among others the following:
solid-state and solution syntheses give the same or closely related products;
solution synthesis gives the desired product, whereas solid state does not; and
solid-state synthesis gives the desired product, but solution does not.

Which of these is the most likely is not yet readily predictable. Mechanochemistry brings its own challenges and sets of rules to synthesis, and many of the latter are not yet fully understood. Mechanism(s) of reactions in the solid state are by no means required to follow those of their solution-based counterparts. Manipulating solid materials introduces different issues of mass transport, and can reduce the effects of steric hindrance to reactivity. These changes can contribute to (as yet) unpredictable patterns of reactivity, whether they involve the promotion of undesired decomposition routes or the generation of products previously believed to be unattainable—see the review paper by Rightmire and Hanusa, Advances in organometallic synthesis with mechanochemical methods, Dalton Trans., 2016, 455, 2352, Abstract, section 3, and conclusion.

Indeed, understanding the fundamental nature of mechanochemical reactions remains an important and largely unsolved problem of mechanochemistry and, in fact, mechanochemical reactions are mostly unpredictable—see the perspective paper by Suslick, Mechanochemistry and sonochemistry: concluding remarks, Faraday Discuss., 2014, 170, 411 on pages 417 and 418.

### SUMMARY OF THE INVENTION

In accordance with the present invention, there is provided:
1. A method for the enzymatic saccharification of a polysaccharide, the method comprising:
   a) the step of contacting the polysaccharide with a hydrolase and water, in the absence of solvent, thereby forming a solid reaction mixture; and
   b) the step of:
      b)-i. mixing and then incubating the solid reaction mixture,
      b)-ii. milling the solid reaction mixture, or
      b)-iii. milling and then incubating the solid reaction mixture.
2. The method of item 1, wherein the polysaccharide is a cellulose, a hemicellulose, chitin, chitosan, starch, glycogen, a pectin, a peptidoglycan, alginate, or a combination thereof, preferably a cellulose, a hemicellulose, chitin or a combination thereof.
3. The method of item 2, wherein the cellulose is cellulose I or microcrystalline cellulose, preferably cellulose I.
4. The method of item 2 or 3, wherein the hemicellulose is xylan.
5. The method of any one of items 1 to 4, wherein the solid reaction mixture has a ratio η of liquid volume, in μL, to total solid weight, in mg, between about 0.01 and about 3 μL/mg, preferably between about 0.01 and about 1.75 μL/mg, more preferably between 0.25 to about 1.75 μL/mg, and most preferably between about 0.6 and about 1.6 μL/mg.
6. The method of any one of items 1 to 5, wherein the polysaccharide comprises a cellulose, a hemicellulose, or a combination thereof.
7. The method of any one of items 1 to 6, wherein the polysaccharide is provided in the form of lignocellulosic biomass.
8. The method of item 7, wherein the lignocellulosic biomass is comminuted prior to step a).
9. The method of any one of items 6 to 8, wherein the hydrolase comprises one or more cellulase, one or more hemicellulase (preferably a xylanase), or a combination thereof, preferably a combination thereof.
10. The method of item 9, wherein the one or more cellulase exhibits two or more, preferably all, of the following types of activity: endocellulase activity, exocellulase activity, and β-glucosidase activity.
11. The method of item 9 or 10, wherein the one or more cellulase is a cellulase from Aspergillus niger or Trichoderma reesei, or Trichoderma longibrachiatum, or a combination thereof.
12. The method of item 9 or 10, wherein the one or more cellulase is a combination of a cellulase from Aspergillus niger, preferably a β-glucosidase from Aspergillus niger, and a cellulase from Trichoderma reesei.
13. The method of item 9 or 10 wherein the one or more cellulase is a cellulase from Trichoderma longibrachiatum.
14. The method of any one of items 9 to 13, wherein the xylanase is a xylanase from Thermomyces lanuginosis.
15. The method of any one of items 1 to 5, wherein the polysaccharide comprises a cellulose
16. The method of item 15, wherein the hydrolase comprise one or more cellulase.
17. The method of item 16, wherein the one or more cellulase exhibits two or more, preferably all, of the following types of activity: endocellulase activity, exocellulase activity, and β-glucosidase activity.
18. The method of item 16 or 17, wherein the one or more cellulase is a cellulase from Aspergillus niger or Trichoderma reesei, or Trichoderma longibrachiatum, or a combination thereof.
19. The method of item 16 or 17, wherein the one or more cellulase is a combination of a cellulase from Aspergillus niger, preferably a β-glucosidase from Aspergillus niger, and a cellulase from Trichoderma reesei.
20. The method of item 16 or 17, wherein the one or more cellulase is a cellulase from Trichoderma longibrachiatum.
21. The method of any one of items 15 to 20, wherein the solid reaction mixture has a ration η of liquid volume, in μL, to total solid weight, in mg, between about 0.01 and about 3 μL/mg, preferably between about 0.01 and about 1.75 μL/mg, more preferably between 0.1 to about 1.5 μL/mg, yet more preferably between about 0.5 and about 1.5 μL/mg, even more preferably between about 0.75 and about 1.25 μL/mg, yet more preferably between about 0.9 and about 1.1 μL/mg, and most preferably is preferably about 1 μL/mg.
22. The method of any one of items 1 to 5, wherein the polysaccharide comprises a hemicellulose, preferably xylan and
23. The method of any one of item 22, wherein the hydrolase comprises a hemicellulase, preferably a xylanase.
24. The method of item 23, wherein the xylanase is a xylanase from Thermomyces lanuginosis.
25. The method of any one of items 22 to 24, wherein the solid reaction mixture has a ratio η of liquid volume, in μL, to total solid weight, in mg, between about 0.01 and about 3 μL/mg, preferably between about 0.01 and about 1.75 μL/mg, more preferably between 0.1 to about 1.5 μL/mg, yet more preferably between about 0.25 and about 1.25 μL/mg, even more preferable between about 0.4 and about 1 μL/mg, yet more preferably between about 0.5 and about 0.7 μL/mg, and most preferably is preferably about 0.6 μL/mg.
26. The method of any one of items 1 to 5, wherein the polysaccharide comprises chitin.
27. The method of item 26, wherein is the chitin is provided as a chitin-containing biomass.
28. The method of item 27, wherein the chitin-containing biomass is comminuted prior to step a).
29. The method of any one of items 26 to 28, wherein the hydrolase comprises a chitinase.
30. The method of item 29, wherein the chitinase is a chitinase from Aspergillus niger, or S. griseus, or Amycolaptosis orientalis.
31. The method of item 30, wherein the chitinase is a chitinase from Aspergillus niger.
32. The method of any one of items 26 to 30, wherein the solid reaction mixture has a ratio η of liquid volume, in μL, to total solid weight, in mg, between about 0.01 and about 3 μL/mg, preferably between about 0.01 and about 1.75 μL/mg, more preferably between 0.1 to about 1.75 μL/mg, yet more preferably between about 0.5 and about 1.75 μL/mg, even more preferable between about 1 and about 1.75 μL/mg, yet more preferably between about 1.5 and about 1.75 μL/mg, and most preferably is preferably about 1.6 μL/mg.
33. The method of any one of items 1 to 32, wherein the hydrolase is a wild type enzyme.
34. The method of any one of items 1 to 33, wherein the hydrolase is a non-immobilized enzyme.
35. The method of any one of items 1 to 34, wherein the solid reaction mixture comprises between about 1V and about 20V of water, preferably between 5V and about 15V, more preferably about 8V to about 12V, and most preferably about 10V of water, V being the volume of the stoichiometric amount of water necessary to achieve a complete hydrolysis of the polysaccharide.
36. The method of any one of items 1 to 35, wherein the solid reaction mixture has a hydrolase concentration of about 0.01 w/w % to about 50 w/w %, preferably between about 0.01 w/w % and about 20 w/w %, more preferably between about 0.01 w/w % and about 5 w/w %, yet more preferably between about 0.05 w/w % and about 4 w/w %, even more preferably between about 0.1 w/w % and about 3 w/w %, and most preferably between about 1 w/w % and about 1.5 w/w %, based on the weight of the polysaccharide.
37. The method of any one of items 1 to 36, wherein in step a), the hydrolase is added to the polysaccharide in dry form and/or in the form of a solution of the hydrolase in water.
38. The method of any one of items 1 to 37, wherein in step a), part or all of, preferably all of, the hydrolase is added to the polysaccharide in dry form.
39. The method of item 38, wherein in step a), the water is added to the polysaccharide separately from the hydrolase, either before or after the hydrolase is added to the polysaccharide.
40. The method of item 38 or 39, wherein in step a), the polysaccharide and the hydrolase are first contacted together and then, the water is added to the polysaccharide and the hydrolase.
41. The method of item 40, wherein the polysaccharide and the hydrolase are further mixed together before the water is added to the polysaccharide and the hydrolase.
42. The method of any one of items 1 to 37, wherein in step a), part or all of, preferably all of, the hydrolase is added to the polysaccharide in the form of a solution of the hydrolase in the water.
43. The method of item 42, wherein further water is added to the solid reaction mixture.
44. The method of any one of items 1 to 43, wherein the water is in the form of pure water or in the form of an aqueous buffer.
45. The method of item 44, wherein the water is in the form of an aqueous buffer.
46. The method of item 44 or 45, wherein the aqueous buffer is a 2-(N-morpholino)ethanesulfonic acid (MES), tris(hydroxymethyl)aminomethane (Tris)-HCl, or a sodium acetate, citrate, phosphate or tartrate buffer, preferably a sodium acetate buffer.
47. The method of any one of items 44 to 46, wherein the aqueous buffer has a pH ranging from about 3 to about 7, preferably from 4.5 to about 7, more preferably from about 5 to about 7, and most preferably a pH of about 5.
48. The method of item 44, wherein the water is in the form of pure water.
49. The method of any one items 1 to 48, wherein the solid reaction mixture further comprises one or more solid additives.
50. The method of item 49, wherein the solid additive is one or more of a powdered salt, a metal or alkaline or alkaline earth oxide, silica beads, silica powder, alumina, polymer beads, or an abrasive powder.
51. The method of any one items 1 to 50, wherein the solid reaction mixture further comprises one or more liquid additives.
52. The method of item 51, wherein the liquid additive is one or more organic liquid, such as ethylene glycol, glycerol, isopropanol, polyethylene glycol of any type or length, a detergent or a polymer such as poly (sorbitol methacrylate).
53. The method of any one items 1 to 52, wherein step b) comprises step b)-ii milling the solid reaction mixture.
54. The method of any one items 1 to 52, wherein step b) comprises step b)-i mixing and then incubating the solid reaction mixture.
55. The method of any one items 1 to 52, wherein step b) comprises step b)-iii milling and then incubating the solid reaction mixture.
56. The method of item 54 or 55, further comprising after step b)-i. or after step b)-iii.:
   the step c) of milling the solid reaction mixture or
   the step c′) of milling and then incubating the solid reaction mixture.
57. The method of item 56, comprising, after step b)-i. or after step b)-iii., preferably after step b)-iii., the step c′) of milling and then incubating the solid reaction mixture.
58. The method of item 57, further comprising after step c′), the step of repeating step c′) one or more times.
59. The method of any one of items 54 to 58, wherein the solid reaction mixture is incubated at a temperature from about 0° C. to about 80° C., preferably from about 20° C. to about 60° C., more preferably from about 30° C. to about 55° C., yet more preferably from about 40° C. to about 50° C., and most preferably about 45° C.
60. The method of any one of items 54 to 59, wherein the solid reaction mixture is incubated under a relative humidity ranging from normal atmospheric conditions to 100% relative humidity, preferably from about 50% to about 100% relative humidity, more preferably from about 75% to about 100% relative humidity, yet more preferably from about 90% to about 100% relative humidity, and more preferably of about 100% relative humidity.
61. The method of any one of items any one of items 54 to 60, wherein the solid reaction mixture is incubated between about 30 minutes and about 30 days, preferably between about 1 hour and about 7 days, and even preferably between about 1 and about 7 days.
62. The method of any one of items 44 and 46 to 52, wherein the solid reaction mixture is milled using a ball mill (including shaker, planetary, attrition, magnetic, and tumbler mills), a roller mill, a knife mill, a mixer mill, a disk mill, a cutting mill, a rotor mill, a pestle mill, a mortar mill, or a kneading trough, preferably a ball mill, more preferably a shaker mill.
63. The method of any one of items 53 and 55 to 62, wherein the solid reaction mixture is milled in a mill at a frequency ranging from about 0.5 to about 100 Hz.
64. The method of any one of items 53 and 55 to 63, wherein the solid reaction mixture is milled in a planetary mill at a frequency from about 3 to about 10 Hz.
65. The method of any one of items 53 and 55 to 64, wherein the solid reaction mixture is milled in a shaker mill at a frequency from about 20 to about 40 Hz, preferably from about 25 to about 35 Hz and more preferably about 30 Hz.
66. The method of any one of items 53 and 55 to 65, wherein the solid reaction mixture is milled in a mixer mill at a frequency from about 60 to about 80 Hz.
67. The method of any one of items 53 and 55 to 66, wherein the solid reaction mixture is milled for 5 min to 90 min, preferably from about 5 to about 60 minutes.
68. The method of any one of items 53 and 55 to 67, wherein the temperature of the solid reaction mixture during milling is of about 80° C. or less, preferably between about 0 to about 80° C., more preferably about 40° C. or less, more preferably between about 20 and about 40° C., and most preferably about room temperature.
69. The method of any one of items 1 to 68, wherein the saccharification produces water-soluble carbohydrates.

### BRIEF DESCRIPTION OF THE DRAWINGS

In the appended drawings:

FIG. 1 shows the digestion of cellulose by sequential action of three enzymes: a) endoglucanase, b) exoglucanase, c) β-glucosidase. Glucose units are represented as gray ellipses.

FIG. 2 shows the results of accelerated ageing between cellulose, a commercial A. niger enzyme preparation, and water.

FIG. 3 shows the influence of volume of liquid used, water (diamonds) or acetate buffer (squares), on reactions between cellulose and a commercial A. niger enzyme preparation.

FIG. 4 shows the percentage of hydrolysis observed as a function of milling time (cellulose, commercial T. reesei enzyme preparation, and water).

FIG. 5 shows the percentage of hydrolysis observed as a function of time as milling/accelerated aging cycles are carried three times a day (cellulose, commercial A. niger enzyme preparation, and water).

FIG. 6 shows the percentage of MCC hydrolysis observed over time for different loadings of T. Longibrachiatum cellulases.

FIG. 7 shows TLC analysis over the reaction mixture after milling and aging of MCC (eluent: EtOAc/MeOH/H₂O 4:2:1.5).

FIG. 8 shows the percentage of MCC hydrolysis by T. longibrachiatum cellulose observed as a function of time for a milling and aging experiment at a larger scale (5 g MCC).

FIG. 9 shows the percentage of MCC hydrolysis observed using recycled enzyme and unreacted MCC in a second round of milling and aging.

FIG. 10 shows the percentage of MCC hydrolysis observed as a function of time when using T. Reesei cellulase alone or T. Reesei cellulase together with A. niger beta-glucosidase (BG).

FIG. 11 shows the percentage of chitin hydrolysis by Aspergillus niger chitinase observed as a function of η for various aging durations.

FIG. 12 shows the percentage of chitin hydrolysis by Aspergillus niger chitinase observed as a function of time after milling, for various enzyme loadings.

FIG. 13 shows the percentage of chitin hydrolysis by Aspergillus niger chitinase observed as a function of enzyme loading when milling alone and when milling is followed by aging for 4 or 7 days.

FIG. 14 shows the percentage of chitin hydrolysis by Aspergillus niger chitinase observed as a function of milling time.

FIG. 15 shows the percentage of chitin hydrolysis by Aspergillus niger chitinase observed as a function of aging time at three temperatures (room temp, 45° C., and 55° C.).

FIG. 16 shows the percentage of xylan hydrolysis by T. lanuginosis xylanase observed under milling (30 Hz, 30 min) as a function of the volume of water used for two xylan sources (either birchwood xylan or oat spelts xylan).

FIG. 17 shows the percentage of xylan hydrolysis by T. lanuginosis xylanase observed for two xylan sources after milling (30 Hz, 30 min) with a η=0.6.

FIG. 18 shows the percentage of birchwood xylan hydrolysis by T. lanuginosis xylanase observed after milling (30 Hz, 30 min) for different enzyme loadings.

FIG. 19 shows the percentage of cellulose hydrolysis by T. longibrachiatum cellulose observed after RAging as a function of time for native sugarcane bagasse (SB) and native wheat straw (WS).

FIG. 20 shows the percentage of cellulose hydrolysis T. longibrachiatum cellulose observed after RAging as a function of time for pre-milled sugarcane bagasse (SB) and pre-milled wheat straw (WS).

FIG. 21 shows the glucose production by T. longibrachiatum cellulose from hay observed using the process of the invention (RAging, columns on the left of each pack), compared to a slurry process in buffer (columns in the middle of each pack) and to a slurry process in water (columns on the right of each pack).

FIG. 22 shows the glucose production by T. longibrachiatum cellulose from cedar tree saw dust observed with the process of the invention (RAging, columns on the left of each pack), compared to a slurry process in buffer (columns in the middle of each pack) and to a slurry process in water (columns on the right of each pack).

FIG. 23 shows the percentage of xylan hydrolysis by T. lanuginosis xylanase observed after milling (30 min, 30 Hz) or milling followed aging (3 days) sugarcane bagasse and wheat straw.

### DETAILED DESCRIPTION OF THE INVENTION

The present invention is based on the unexpected discovery that enzymes can be used to catalyze a chemical reaction, more specifically the saccharification of a polysaccharide, under solvent-free conditions and that this allows the enzymes to work on otherwise inaccessible, low solubility polysaccharides, such as cellulose.

The invention is also based on the unexpected discovery that milling the solvent-free reaction mixture does not deactivate the enzymes but, in fact, speeds up and increases the yield of the hydrolysis reaction.

### Polysaccharide & Saccharification

Turning now to the invention in more details, there is provided a method for the enzymatic saccharification of a polysaccharide.

Herein, a polysaccharide is a polymeric carbohydrate molecule composed of long chains of monosaccharide units bound together by glycosidic bonds. A non-limiting example of polysaccharide is cellulose, which is made of glucose monosaccharide units bound together by glycosidic bonds:

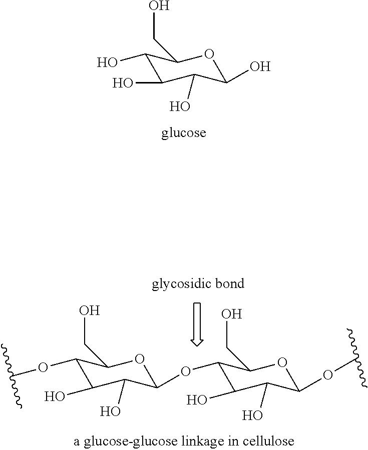 [SMILES: OCC1OC(O)C(O)C(O)C1O ; *OC1OC(CO)C(OC2OC(CO)C(O*)C(O)C2O)C(O)C1O]

The saccharification of a polysaccharide is the breakdown, or depolymerisation, of the polysaccharide into oligosaccharides and/or its constituting monosaccharide units. Oligosaccharides are similar to the polysaccharide, except that they are constituted of shorter chains of the monosaccharide units. The breakdown of the polysaccharide during saccharification occurs via hydrolysis. More specifically, the glycosidic bonds of the polysaccharide are cleaved by the addition of a water molecule:

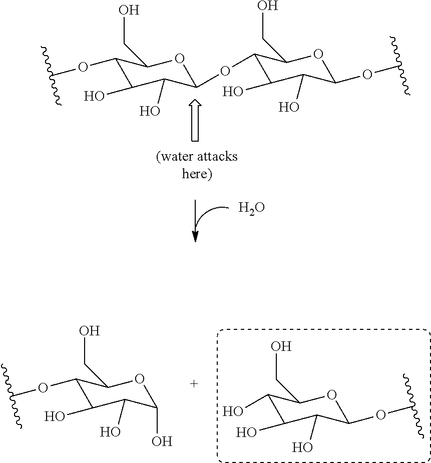 [SMILES: *OC1OC(CO)C(OC2OC(CO)C(O*)C(O)C2O)C(O)C1O ; O ; *OC1C(CO)OC(O)C(O)C1O ; *OC1OC(CO)C(O)C(O)C1O]

In saccharification, the hydrolytic decomposition of the polysaccharide is achieved by the presence of a catalyst. While various catalysts are known, the method of the invention is limited to enzymatic saccharification, that is saccharification using enzymes, called hydrolases or hydrolytic enzymes, as catalysts for hydrolysis of the polysaccharide.

The saccharification may be complete or partial. In complete saccharification, the polysaccharide is broken down into its constituting monosaccharide units with few or no remaining oligosaccharides. In partial saccharification, polysaccharide is broken down into its constituting monosaccharide units and oligosaccharides. The completeness of the saccharification is expressed as a conversion rate representing the percentage of the free monosaccharide units cleaved off the polysaccharide. A method for measuring the conversion rate is presented in Example 1 below.

Generally speaking, higher conversion rates are preferred. However, complete saccharification is not necessary. Rather, for most applications, it is often desired to simply break-down an insoluble polysaccharide into monosaccharide units and/or oligosaccharides that are soluble (preferably herein (in)solubility refers to (in)solubility in water), so that they can be further processed into other commercial products (e.g. ethanol, succinic acid, furfural, etc.). In such cases, saccharification via the method of the invention simply aims to transform the polysaccharide into products that are amenable to such known processing. Thus, in an embodiment of the invention, the enzymatic saccharification of the polysaccharide, especially a water-insoluble polysaccharide, yields water-soluble monosaccharide units and/or oligosaccharides, which can be collectively referred to as water-soluble carbohydrates.

The polysaccharide used as a feedstock for the method of the invention can be of various nature. Non-limiting examples of polysaccharides include celluloses, hemicelluloses, chitin, chitosan, starch, glycogen, pectins, peptidoglycans, alginate, and combinations thereof. Preferred polysaccharides include celluloses, hemicelluloses, chitin, and combinations thereof. More preferred polysaccharides include celluloses, hemicelluloses, and combinations thereof. Alternative more preferred polysaccharides include chitin.

As noted above, cellulose is a linear polysaccharide composed of β(1→4) linked D-glucose units.

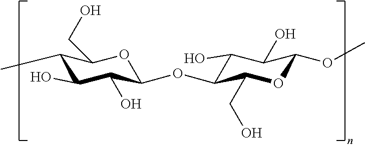

Cellulose is the main component of the cellular walls of higher plants. It has a complex supramolecular structure resulting from the ordering and association of its molecules. The multiple hydroxyl groups on the glucose from one chain form hydrogen bonds with oxygen atoms on the same or on a neighboring chain, holding the chains firmly together side-by-side and forming primary fibrils, which are held together by further hydrogen bonds, thus forming microfibrils. The cellulose macromolecules in the microfibrils form highly ordered crystalline zones that alternate with inhomogeneous, less ordered amorphous zones. Such specific cellulose morphological structure makes it stable when exposed to significant mechanical loads. Furthermore, cellulose is quite stable to enzymes and microorganisms. These challenges arise primarily because “plants have evolved to be recalcitrant to attack by the elements, and in particular by microbes and their enzymes”—see Olson et al., Curr. Opin. Biotech. 2012, 23, 396-405. Several different crystalline structures of cellulose are known, corresponding to the location of hydrogen bonds between and within strands. Natural cellulose is cellulose I, with structures I_{α} and I_{β}. Cellulose produced by bacteria and algae is enriched in I_{α} while cellulose of higher plants consists mainly of I_{β}. Cellulose in regenerated cellulose fibers is cellulose II. With various chemical treatments it is possible to produce the structures cellulose III and cellulose IV. Cellulose in all its forms can be suitably used as a feedstock in the present invention. Such forms of cellulose include: cellulose I (including cellulose I_{α} and cellulose I_{β}), cellulose II, cellulose III, cellulose IV, amorphous cellulose (obtained using high temperature and pressure), nanocrystalline cellulose (obtained by treatment with a strong acid that breaks up the amorphous regions can in the cellulose), microcrystalline cellulose (pure partially depolymerized cellulose synthesized from α-cellulose precursor), etc. Chemically modified variations of cellulose can also be used, for example sulfonated, carboxylated, phosphorylated, acetylated. A preferred cellulose is cellulose I or microcrystalline cellulose, preferably cellulose I.

A hemicellulose (also known as polyose) is any of several heteropolysaccharides present along with cellulose in almost all plant cell walls. While cellulose is crystalline, strong, and resistant to hydrolysis, hemicellulose has a random, amorphous structure with less strength. It can typically be hydrolyzed by dilute acid or base, as well as hemicellulase enzymes. Hemicelluloses include xylan, glucuronoxylan, arabinoxylan, glucomannan, and xyloglucan. These polysaccharides contain many different monosaccharide units. In contrast, cellulose contains only glucose. For instance, besides glucose, monosaccharide units in hemicellulose can include xylose, mannose, galactose, rhamnose, and arabinose. Hemicelluloses contain most of the D-pentose sugars, and occasionally small amounts of L-sugars as well. The monosaccharide units are usually combined by β-1,4-links, the latter having frequently lateral links of another type. A preferred hemicellulose is xylan.

Both cellulose and hemicellulose are found in lignocellulose. Lignocellulose refers to plant dry matter (biomass), also called lignocellulosic biomass. In preferred embodiments, the polysaccharide of the method of the invention is provided in the form of lignocellulosic biomass. Lignocellulosic biomass is the most abundantly available raw material on the Earth for the production of biofuels, mainly bio-ethanol. Lignocellulose is composed of cellulose, hemicellulose, and lignin (an aromatic polymer). When lignocellulose is used as a feedstock in the method of the invention, its amorphous cellulose and hemicellulose parts are hydrolyzed, yielding water-soluble carbohydrates, leaving lignin. The lignocellulose can be comminuted (i.e. reduced into smaller particles) before being used as feedstock. For example, the lignocellulose can be milled for a few minutes.

Lignocellulose feedstocks suitable for this method include, without limitations, the following types: agricultural plants, hay, corn stocks, corn ears, wheat, oat straw, rice straw, sugarcane stocks (bagasse), flax straw (boon), soy bean stems, groundnut stems, pea stems, sugar beet stems, sorghum stems, tobacco stems, maize, barley straw, buckwheat straw, cassava stems, potato stems, bean stems, cotton and its stems, inedible parts of plants, grain shells (husk); wood of fir, pine, silver fir, cider, cedar, larch, oak, ash, birch, aspen, poplar, beech, maple, nut-tree, cypress, elm, chestnut, alder, hickory, acacia, plane tree, pepperidge, butternut, apple tree, pear tree, plum tree, cherry tree, cornel, catalpa, boxtree, camphor tree, redwood, lanceolate oxandra, tall mora, primavera, rose tree, teak wood, satinwood, mangrove wood, orange-wood, lemon, logwood, scumpia, orange maclura, hedge wood cisalpine, fragrant cisalpine, camwood, sandalwood, rubber-bearing wood, huta, mesquite, eucalyptus, shrubs, oleander, cypress, juniper, acanthus, lantana, bougainvillea, azalea, feijoa, holly, hibiscus, stramonium, acutifolia, hydrangea, jasmine, rhododendron, common Palma Christi, myrtle, euonymus, aralias, algae, brown algae, herbs, creeping plants, common grass and flowers.

Other sources of cellulose that can be used as feedstock include commercial waste containing cellulose, such as paper, recycled paper, cotton fabric, and timber, as well as partially decomposed vegetable materials, such as mowed grass.

Chitin is the most abundant nitrogen-containing biopolymer on the planet. It is a linear polysaccharide composed of units of 2-(acetylamino)-2-deoxy-D-glucose, which is a derivative of glucose. These units form covalent β-(1→4)-linkages, similar to the linkages between the glucose units forming cellulose. Therefore, chitin may be described as cellulose with one hydroxyl group on each monomer replaced with an acetyl amine group. Chitin is found in many places throughout the natural world. It is a characteristic component of the cell walls of fungi, the exoskeletons of arthropods (such as crustaceans) and insects, the radulae of molluscs, the beaks and internal shells of cephalopods, and on the scales and other soft tissues of fish and lissamphibians.

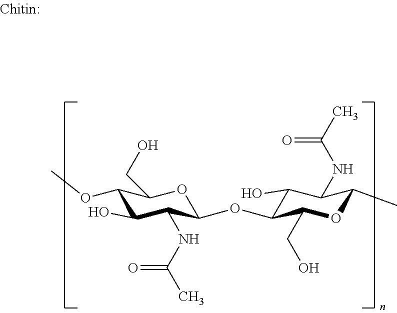

Chitin can be provided in the form of a chitin-containing biomass. The chitin-containing biomass that can be used as feedstock for the method of the invention include crustacean shells, for example shrimp shells, crab shells, and lobster shells, preferably provided as byproducts of the food-processing industry. The chitin-containing biomass can be comminuted (i.e. reduced into smaller particles) before being used as feedstock. For example, the chitin-containing biomass can be milled for a few minutes.

Chitosan is a linear polysaccharide composed of randomly distributed β-(1→4)-linked D-glucosamine (deacetylated unit) and 2-(acetylamino)-2-deoxy-D-glucose (acetylated unit). It is made by deacetylating chitin. The deacetylation may be complete or partial.

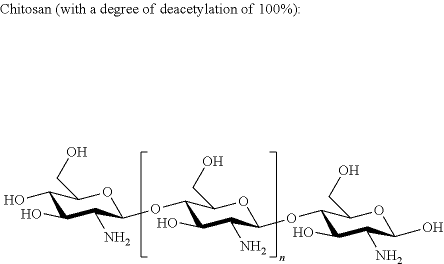

Starch (or amylum) is a polymeric carbohydrate consisting of a large number of glucose units joined by glycosidic bonds. This polysaccharide is produced by most green plants as an energy store. It consists of two types of molecules: the linear and helical amylose and the branched amylopectin. Depending on the plant, starch generally contains 20 to 25% amylose and 75 to 80% amylopectin by weight. Amylose is a helical polymer made of α-D-glucose units, bound to each other through α(1→4) glycosidic bonds. Amylopectin is a soluble polysaccharide and highly branched polymer of glucose. Its glucose units are linked in a linear way with α(1→4) glycosidic bonds. Branching takes place with α(1→6) bonds occurring every 24 to 30 glucose units. In contrast, amylose contains very few 60 (1→6) bonds, or even none at all.

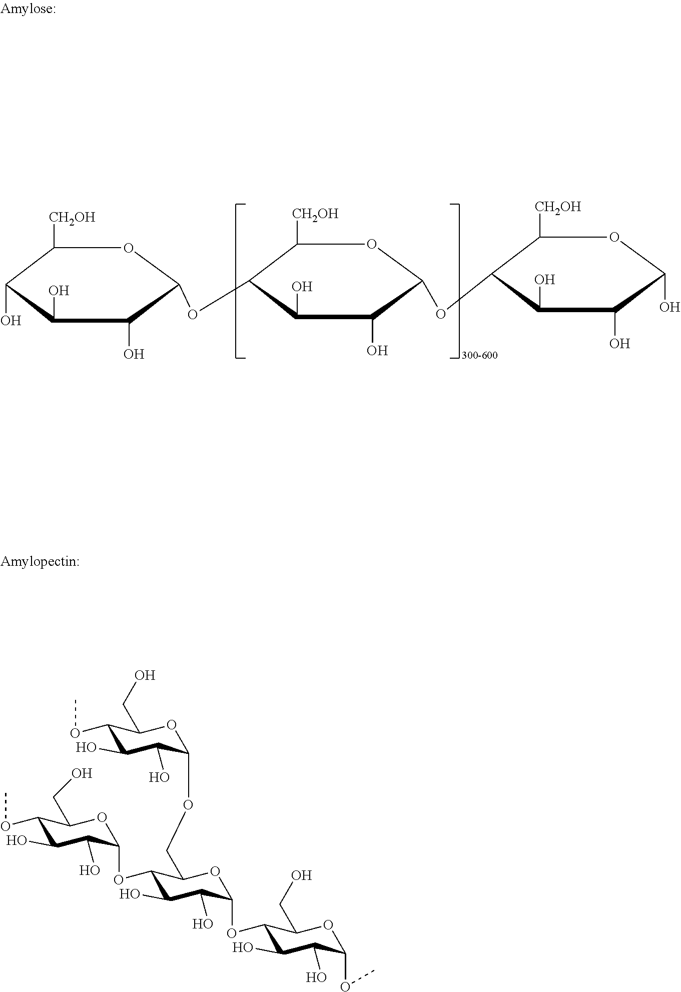

Glycogen is a multi-branched polysaccharide of glucose that serves as a form of energy storage in humans, animals, insects and fungi. The polysaccharide structure represents the main storage form of glucose in the body. Glycogen is the analogue of starch, a glucose polymer that functions as energy storage in plants. It has a structure similar to amylopectin (a component of starch), but is more extensively branched and compact than starch. More specifically, glycogen is a branched biopolymer consisting of linear chains of glucose units with further chains branching off every 8 to 12 glucose units or so. Glucose units are linked together linearly by α(1→4) glycosidic bonds from one glucose to the next. Branches are linked to the chains from which they are branching off by α(1→6) glycosidic bonds between the first glucose of the new branch and a glucose on the stem chain.

Glycogen and its Chemical Structure:

Pectins form a group of structural heteropolysaccharides contained in the primary cell walls of terrestrial plants. Pectins, also known as pectic polysaccharides, are rich in galacturonic acid. Several distinct polysaccharides have been identified and characterised within the pectic group. Homogalacturonans are linear chains of α-(1-4)-linked D-galacturonic acid. Substituted galacturonans are characterized by the presence of saccharide appendant residues (such as D-xylose or D-apiose in the respective cases of xylogalacturonan and apiogalacturonan) branching from a backbone of D-galacturonic acid residues. Rhamnogalacturonan I pectins (RG-I) contain a backbone of the repeating disaccharide: 4)-α-D-galacturonic acid-(1,2)-α-L-rhamnose-(1. From many of the rhamnose residues, sidechains of various neutral sugars branch off. The neutral sugars are mainly D-galactose, L-arabinose and D-xylose, with the types and proportions of neutral sugars varying with the origin of pectin. Another structural type of pectin is rhamnogalacturonan II (RG-II), which is a less frequent, complex, highly branched polysaccharide.

Peptidoglycan, also known as murein, is a polymer consisting of sugars and amino acids that forms a mesh-like layer outside the plasma membrane of most bacteria, forming the cell wall. The sugar component consists of alternating residues of β-(1,4) linked N-acetylglucosamine and N-acetylmuramic acid. Attached to the N-acetylmuramic acid is a peptide chain of three to five amino acids. The peptide chain can be cross-linked to the peptide chain of another strand forming the 3D mesh-like layer.

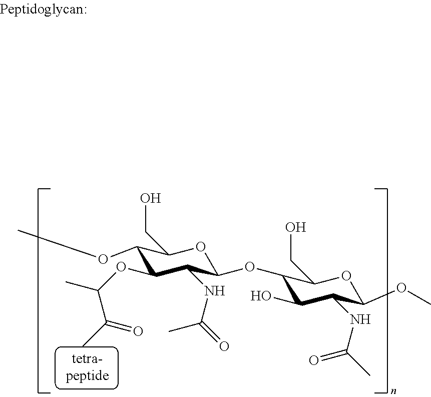

Alginic acid, also called algin or alginate, is an anionic polysaccharide distributed widely in the cell walls of brown algae, where through binding with water it forms a viscous gum. It is also a significant component of the biofilms produced by the bacterium. Alginic acid is a linear copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks) or alternating M and G-residues (MG-blocks).

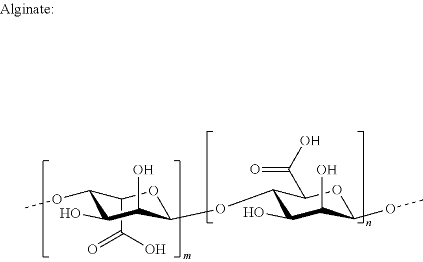

### Step a)

The method of the invention first comprises a) the step of contacting the polysaccharide with a hydrolase and water, in the absence of solvent, thereby forming a solid reaction mixture.

Indeed, to effect saccharification, the polysaccharide is contacted with a hydrolase, i.e. a hydrolytic enzyme, that will act as a catalyst for the hydrolysis of the polysaccharide. Indeed, a hydrolase or hydrolytic enzyme is an enzyme that catalyzes the hydrolysis of a chemical bond.

In embodiments, the hydrolase is a wild type or native enzyme, which has the advantage of being less costly than other alternatives. The hydrolase may be isolated from natural sources (e.g., bacteria, fungi, plants) or may be produced recombinantly in a suitable host cell (e.g., E. coli). In other embodiments, the hydrolase can also be a mutated enzyme.

The hydrolase is preferably non-immobilized. In other words, it is not attached to a solid support. In other embodiments, the hydrolase is immobilized.

The Enzyme Commission number (EC number) is a numerical classification scheme for enzymes, based on the chemical reactions they catalyze. Every enzyme code consists of the letters “EC” followed by four numbers separated by periods. Those numbers represent a progressively finer classification of the enzyme. Hydrolases form the EC 3 class of this classification system.

The exact hydrolase used will be selected according to the product required and/or feedstock used. For a given feedstock and/or a desired product, a mixture of hydrolases can be used if desired. For example, the treatment of lignocellulosic biomass may advantageously use a combination of a cellulase and a hemicellulase (see below for details).

Also, when the process is applied to a mixture of feedstocks, a mixture of hydrolases, each selected according to one or more of the feedstocks presents, is advantageously used.

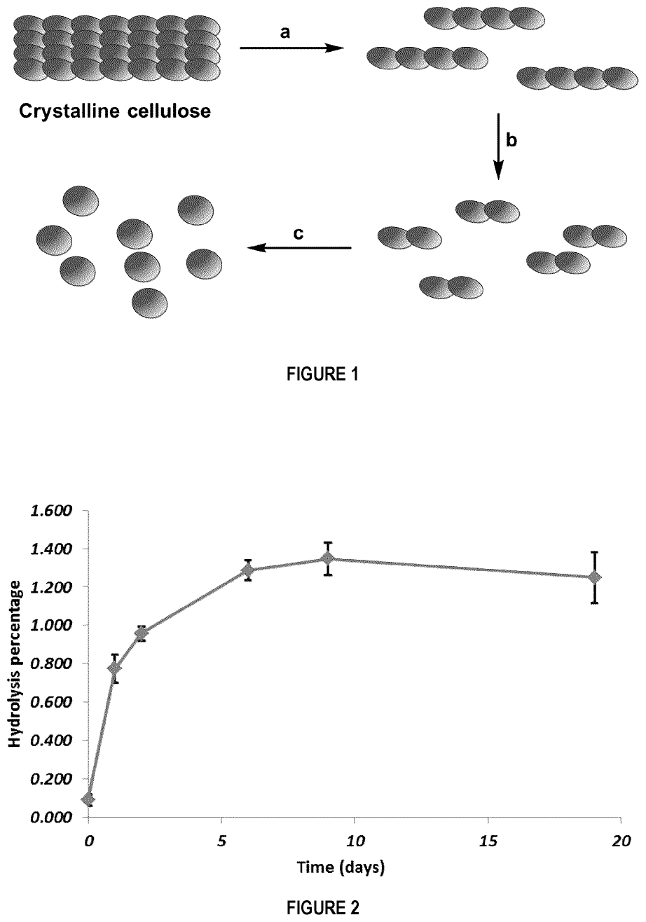

Celluloses are hydrolysed by cellulases. Cellulase activity encompasses a set of three elemental enzymatic actions described in FIG. 1. These three types of cellulases/activity are preferably used together in the method of the invention:
Endocellulases (also called endoglucanases, endopolymerases, endoglucanases, endoenzymes, EC 3.2.1.4) are responsible for the breaking of cellulose strands into oligosaccharides. They randomly cleave internal bonds to create new chain ends. They hydrolyze effectively internal glycoside links between monosaccharide units.
Exocellulases (also called cellobiohydrolases, exodepolymerase, exogluconases, exoenzymes, EC 3.2.1.91) split preferably the terminal and/or sub-terminal glycoside links at the ends of the polysaccharide chain. They cleave two to four units from the ends of the exposed chains produced by endocellulase, resulting in tetrasaccharides or disaccharides (cellobiose).
Cellobiases (EC 3.2.1.21) or β-glucosidases hydrolyse the exocellulase product into individual monosaccharides by performing hydrolysis of the glycoside links of di- and oligosaccharides.

Most commercially available cellulase enzymes are constituted of a mix of several cellulases and display one, two or three of the above activities. As non-limiting examples, we list below some cellulases available from Sigma-Aldrich®:

| Description | EC/CAS/Sigma |
|---|---|
| (Details on activity) | Aldrich no. |
| Cellulase from Aspergillus niger | 3.2.1.4/9012-54-8/C1184 & |
| (catalyzes the hydrolysis of endo-1,4-β-D-glycosidic linkages in | 22178 |
| cellulose, lichenin, barley glucan, and the cellooligosaccharides | |
| cellotriose to cellohexaose) | |
| Cellulase from Aspergillus sp. Carezyme ® 1000L | 3.2.1.4/9012-54-8/C2605 |
| (hydrolyzes cellulose, a linear polymer of anhydroglucose units linked | |
| together by β-1,4-glycosidic bonds, to glucose) | |
| Cellulase from Trichoderma longibrachiatum | 3.2.1.4/9012-54-8/C9748 |
| (with xylanase, pectinase, mannanase, xyloglucanase, laminarase, β- | |
| glucosidase, β-xylosidase, α-L-arabinofuranosidase, amylase, and | |
| protease activities) | |
| Cellulase from Trichoderma reesei ATCC 26921 | 3.2.1.4/9012-54-8/C8546 |
| (hydrolyzes cellulose, a linear polymer of anhydroglucose units | |
| linked together by β-1,4-glycosidic bonds, to glucose) | |
| Cellulase from Trichoderma reesei ATCC 26921 | 3.2.1.4/9012-54-8/C2730 |
| Celluclast ® 1.5L | |
| (hydrolyzes cellulose, a linear polymer of anhydroglucose units linked | |
| together by β-1,4-glycosidic bonds, to glucose) | |
| Cellulase from Trichoderma sp. Onozuka RS | 3.2.1.4/9012-54-8/C0615 |
| (hydrolyze cellulose to glucose) | |
| Cellulase from Trichoderma sp. | 3.2.1.4/9012-54-8/C1794 |
| (promotes the endohydrolysis of (1->4)-beta-D-glucosidic linkages in | |
| cellulose and lichenin) | |
| Cellulase, thermostable from Clostridium thermocellum, recombinant, | 3.2.1.4/9012-54-8/C9499 |
| expressed in E. coli | |
| (hydrolyzes cellulose to glucose) | |
| endo-1,4-β-D-glucanase from Acidothermus cellulolyticus, recombinant, | 3.2.1.4/NA/E2164 |
| expressed in corn | |
| Cellobiohydrolase I from Hypocrea jecorina, recombinant, expressed in | 3.2.1.91/NA/E6412 |
| corn | |
| (Cellobiohydrolase is a cellulase which degrades cellulose by | |
| hydrolysing the 1,4-β-D-glycosidic bonds, can be used in combination | |
| with endocellulases and b-glucosidase to produce glucose from | |
| cellulose.) | |
| β-Glucosidase from almonds | 3.2.1.21/9001-22-3/G4511 & |
| (hydrolysis of β-glycosidic bonds connecting carbohydrate residues in | G0395 & 49290 |
| β-D-glycosides. Convert cellobiose and cellooligosaccharides produced | |
| by the endo and exoglucanases to glucose.) | |
| β-Glucosidase, thermostable, recombinant, expressed in E. coli | NA/9001-22-3/G8798 |
| (breaks β1->4 bonds that link oligosaccharides.) | |
| Cellulase, enzyme blend, Cellic CTec2 ® | NA/NA/SAE0020 |
| (cellulase, ß-glucosidase, and hemicellulase activities) | |
| Viscozyme ®, cellulolytic enzyme preparation, Cell Wall Degrading | NA/NA/V2010 |
| Enzyme Complex from Aspergillus sp., Lysing Enzyme from Aspergillus | |
| sp., Multi-enzyme complex containing a wide range of carbohydrases, | |
| including arabanase, cellulase, β-glucanase, hemicellulase, and | |
| xylanase | |
| Driselase ® from Basidiomycetes sp., a mixture of cell wall degrading | NA/85186-71-6/D9515 or |
| enzymes that contains laminarinase, xylanase and cellulase. | D8037 |
| Pectinase from Rhizopus sp., Macerozyme ® R-10, Poly-(1,4-α-D- | 3.2.1.15/9032-75-1/P2401 |
| galacturonide) glycanohydrolase, | |
| (has pectinase activity, as well as cellulase and hemicellulase activities) | |
| Pectinase from Aspergillus niger, Poly-(1,4-α-D-galacturonide) | 3.2.1.15/9032-75-1/P4716 |
| glycanohydrolase, | |
| (has pectinase activity, as well as cellulase and hemicellulase activities) | |
| Pectinase from Aspergillus aculeatus, Pectinex ® Ultra SPL, | NA/NA/P2611 |
| (has pectinase activity, as well as cellulase and hemicellulase activities) | |
| Cellulase from Trichoderma longibrachiatum | 3.2.1.4/9012-54-8/C9748 |
| (with xylanase, pectinase, mannanase, xyloglucanase, laminarase, β- | |
| glucosidase, β-xylosidase, α-L-arabinofuranosidase, amylase, and | |
| protease activities) | |
| Glucosidase from Aspergillus niger | NA/9033-06-1/49291 |
| (Glucosidase catalyzes the hydrolysis of α-1,4 linkages with a substrate | |
| preference for maltose, maltotriose and maltotetraose. Reactivity with | |
| large polysaccharides like dextrin and starch have also been | |
| described.) | |

* Enzymes in boldface are preferred.

Preferred cellulases include those from Aspergillus niger, Trichoderma reesei, or Trichoderma longibrachiatum and combinations thereof, more preferably cellulases from Trichoderma longibrachiatum, or alternatively a combination of a cellulase from Aspergillus niger and a cellulase from Trichoderma reesei.

In embodiments when the feedstock is cellulose, a mixture of two or more cellulases, and more specifically two or more of the three types of cellulases, is preferably used.

Hemicelluloses are hydrolysed by hemicellulases. Hemicellulases are often found in combination with amylase, glucanase, or cellulase. Enzymes that hydrolyse a specific type of hemicellulose can bear a name that relates to this type of hemicellulose (e.g. xylan/xylanase). As non-limiting examples, we list below some hemicellulases available from Sigma-Aldrich®:

| Description | EC/CAS/Sigma |
|---|---|
| (Details on activity) | Aldrich no. |
| Hemicellulase from Aspergillus niger, using a β-galactose | NA/9025-56-3/H2125 |
| dehydrogenase system and locust bean gum as substrate | |
| Xylanase, recombinant, expressed in Aspergillus oryzae, | NA/37278-89-0/X2753 |
| Pentopan Mono BG ® | |
| (endo-β-(1→4)-xylanase) | |
| Xylanase from Trichoderma viride | 3.2.1.8/9025-57-4/X3876 |
| Cellulase, enzyme blend, Cellic CTec2 ® | NA/NA/SAE0020 |
| (cellulase, ß-glucosidase, and hemicellulase activities) | |
| Viscozyme ®, cellulolytic enzyme preparation from Aspergillus | NA/NA/V2010 |
| sp., containing a wide range of carbohydrases, including | |
| arabanase, cellulase, β-glucanase, hemicellulase, and | |
| xylanase | |
| Driselase ® from Basidiomycetes sp., a mixture of cell wall | NA/85186-71-6/D9515 or |
| degrading enzymes that contains laminarinase, xylanase and | D8037 |
| cellulase. | |
| Pectinase from Rhizopus sp., Macerozyme ® R-10, Poly-(1,4- | 3.2.1.15/9032-75-1/P2401 |
| α-D-galacturonide) glycanohydrolase | |
| (has pectinase activity, as well as cellulase and hemicellulase | |
| activities) | |
| Pectinase from Aspergillus niger, Poly-(1,4-α-D-galacturonide) | 3.2.1.15/9032-75-1/P4716 |
| glycanohydrolase | |
| (has pectinase activity, as well as cellulase and hemicellulase | |
| activities) | |
| Pectinase from Aspergillus aculeatus, Pectinex ® Ultra SPL | NA/NA/P2611 |
| (has pectinase activity, as well as cellulase and hemicellulase | |
| activities) | |
| endo-1,4-β-Xylanase from Trichoderma longibrachiatum, | 3.2.1.8/NA/X2629 |
| (Primary activity is an acid-neutral endo-1,4-β-D-xylanase, | |
| additional activities include β-glucanase, cellulase, pectinase, | |
| mannanase, xyloglucanase, laminarase, β-glucosidase, β- | |
| xylosidase, α-L-arabinofuranosidase, amylase, and protease.) | |
| Xylanase 1, thermostable, recombinant, expressed in E. coli | NA/9025-57-4/X3254 |
| Xylanase 2, thermostable, recombinant, expressed in E. coli | NA/9025-57-4/X3379 |
| β-Glucanase 1, thermostable, recombinant, expressed in E. coli, | NA/62213-14-3/G8548 |
| (exhibits endo-xylanase, arabinoxylanase, β-xylosidase and β- | |
| glucosidase activities) | |
| Cellulase from Trichoderma longibrachiatum | 3.2.1.4/9012-54-8/C9748 |
| (with xylanase, pectinase, mannanase, xyloglucanase, | |
| laminarase, β-glucosidase, β-xylosidase, α-L- | |
| arabinofuranosidase, amylase, and protease activities) | |

In embodiments, the hemicellulase is a xylanase, preferably a xylanase from Thermomyces lanuginosis.

Chitin is hydrolysed by chitinases, which break down glycosidic bonds in chitin. Chitinases (EC 3.2.1.14) include chitodextrinase, 1,4-β-poly-N-acetylglucosaminidase, poly-β-glucosaminidase, β-1,4-poly-N-acetyl glucosamidinase, poly[1,4-(N-acetyl-β-D-glucosaminide)] glycanohydrolase, and (1→4)-2-acetamido-2-deoxy-β-D-glucan glycanohydrolase. Chitinases are generally found in organisms that either need to reshape their own chitin or dissolve and digest the chitin of fungi or animals. Chitinases are also present in plants. As non-limiting examples, we list below some chitinases available from Sigma-Aldrich®:

| Description | EC/CAS/Sigma Aldrich no. |
|---|---|
| Chitinase from Streptomyces griseus | 3.2.1.14/9001-06-3/C6137 |
| Chitinase from Trichoderma viride | 3.2.1.14/NA/C8241 |

In embodiments, the hydrolase is a chitinase, preferably a chitinase from Aspergillus niger, or from S. griseus, or from Amycolaptosis orientalis, and more preferably a chitinase from Aspergillus niger.

Chitosan is hydrolysed by chitosanases, also called chitosan N-acetylglucosaminohydrolase, which catalyse the endohydrolysis of beta-(1→4)-linkages between D-glucosamine residues in chitosan. As non-limiting examples, we list below some chitosanases available from Sigma-Aldrich®:

| Description | EC/CAS/Sigma |
|---|---|
| (Details on activity) | Aldrich no. |
| Chitosanase from Streptomyces griseus | 3.2.1.132/51570-20-8/C9830 |
| Chitosanase from Streptomyces sp. | 3.2.1.132/51570-20-8/C0794 |

Both starch and glycogen are hydrolysed by amylases, which catalyse their hydrolysis into sugars. Amylase is present in the saliva of humans and some other mammals, where it begins the chemical process of digestion. Plants and some bacteria also produce amylase. Specific amylase proteins are designated by different Greek letters. All amylases are glycoside hydrolases and act on α-1,4-glycosidic bonds. α-Amylase (also called 1,4-α-D-glucan glucanohydrolase or glycogenase, EC 3.2.1.1) hydrolyses alpha bonds in large, alpha-linked polysaccharides, such as starch and glycogen, yielding glucose and maltose. β-Amylase (also called also called 1,4-α-D-glucan-maltohydrolase or glycogenase, EC 3.2.1.2) acts on starch, glycogen and related polysaccharides and oligosaccharides producing beta-maltose by an inversion. In fact, working from the non-reducing end, β-amylase catalyzes the hydrolysis of the second α-1,4 glycosidic bond, cleaving off two glucose units (maltose) at a time. γ-Amylase (also called glucan 1,4-α-glucosidase, EC 3.2.1.3) will cleave α(1-6) glycosidic linkages, as well as the last α(1-4)glycosidic linkages at the non-reducing end of amylose and amylopectin, yielding glucose. As non-limiting examples, we provide list below some amylases available from Sigma-Aldrich®:

| Description | EC/CAS/Sigma Aldrich no. |
|---|---|
| α-Amylase from porcine pancreas | 3.2.1.1/NA/A3176, |
| | A6255 & A4268 |
| α-Amylase from Bacillus licheniformis | 3.2.1.1/9000-85-5/A3403, |
| | A4582, A4551, 10067 & |
| | A4862 |
| α-Amylase from Aspergillus oryzae | 3.2.1.1/9001-19-8/10065, |
| | A8220, 86250 & A9857 |
| α-Amylase from Bacillus licheniformis, | 3.2.1.1/9000-85-5/A3306 |
| heat-stable | |
| α-Amylase from Bacillus amyloliquefaciens | 3.2.1.1/9000-85-5/A7595 |
| α-Amylase from human saliva | 3.2.1.1/9000-90-2/A1031 & |
| | A0521 |
| β-Amylase from barley | 3.2.1.2/9000-91-3/A7130 |
| α-Amylase from human pancreas | 3.2.1.1/9000-90-2/A9972 |
| α-Amylase from pig pancreas | 3.2.1.1/NA/10102814001 |
| | ROCHE |

Pectins are broken down using pectinases. Commonly referred to as pectic enzymes, pectinases include pectolyase (or pectin lyase), pectozyme, and polygalacturonase.

Pectolyase ((1→4)-6-O-methyl-α-D-galacturonan lyase, EC 4.2.2.10) is a class of naturally occurring pectinase. It is produced commercially for the food industry from fungi and used to destroy residual fruit starch, known as pectin, in wine and cider. Pectin lyase is an enzyme that catalyzes the eliminative cleavage of (1→4)-α-D-galacturonan methyl ester to give oligosaccharides with 4-deoxy-6-O-methyl-α-D-galact-4-enuronosyl groups at their non-reducing ends.

Polygalacturonase (EC 3.2.1.15), also known as pectin depolymerase, PG, pectolase, pectin hydrolase, and poly-alpha-1,4-galacturonide glycanohydrolase, is an enzyme that hydrolyzes the alpha-1,4 glycosidic bonds between galacturonic acid residues. Polygalacturonan, whose major component is galacturonic acid, is a significant carbohydrate component of the pectin network that comprises plant cell walls.

As non-limiting examples, we list below some pectinases available from Sigma-Aldrich®:

| Description | EC/CAS/Sigma |
|---|---|
| (Details on activity) | Aldrich no. |
| Pectinase from Aspergillus niger | 3.2.1.15/9032-75-1/ |
| | P4716, P0690 & 17389 |
| Pectinase from Rhizopus sp. | 3.2.1.15/9032-75-1/ |
| | P2401 & 76287 |
| Pectinase from Aspergillus aculeatus | NA/NA/P2611 & |
| | E6287 |
| Pectolyase from Aspergillus japonicus | 3.2.1.15/NA/P3026 & |
| | P5936 |
| Driselase ® from Basidiomycetes sp., | NA/85186-71-6/ |
| a mixture of cell wall degrading enzymes that | D9515 or D8037 |
| contains laminarinase, xylanase and cellulase. | |
| Pectinase from Aspergillus niger | 3.2.1.15/9032-75-1/ |
| | P4716, P0690 & 17389 |
| Pectinase from Rhizopus sp. | 3.2.1.15/9032-75-1/ |
| | P2401 & 76287 |
| Pectinase from Aspergillus aculeatus | NA/NA/P2611 & |
| | E6287 |
| Pectolyase from Aspergillus japonicus | 3.2.1.15/NA/P3026 & |
| | P5936 |
| Pectinase from Rhizopus sp., Macerozyme ® | 3.2.1.15/9032-75-1/ |
| R-10, Poly-(1,4-α-D-galacturonide) | P2401 |
| glycanohydrolase, has pectinase activity, also | |
| containing cellulase and hemicellulase activities | |
| Pectinase from Aspergillus niger, Poly-(1,4-α- | 3.2.1.15/9032-75-1/ |
| D-galacturonide) glycanohydrolase, has | P4716 |
| pectinase activity, also containing cellulase | |
| and hemicellulase activities | |
| Pectinase from Aspergillus aculeatus, | NA/NA/P2611 |
| Pectinex ® Ultra SPL, has pectinase activity, | |
| also containing cellulase and hemicellulase | |
| activities. | |

Peptidoglycans are hydrolyzed by lysozymes. Lysozymes, also known as muramidase or N-acetylmuramide glycanhydrolase, are glycoside hydrolases. These are enzymes (EC 3.2.1.17) that catalyze hydrolysis of 1,4-beta-linkages between N-acetylmuramic acid and the fourth carbon atom of N-acetyl-D-glucosamine residues in peptidoglycans.

As non-limiting examples, we list below lysozymes available from Sigma-Aldrich®:

| Description | EC/CAS/Sigma |
|---|---|
| (Details on activity) | Aldrich no. |
| Lysozyme from chicken egg white | 3.2.1.17/12650-88-3/L6876, |
| | 62970, 62971, L7651 & L7773 |
| Lysozyme human recombinant, | 3.2.1.17/12671-19-1/L1667 |
| expressed in rice | |
| Lysozyme chloride form from chicken | 3.2.1.17/9066-59-5/L2879 |
| egg white | |
| Lysozyme from human neutrophils | 3.2.1.17/9001-63-2/L8402 |
| Lysozyme, Chicken Egg White; Native, | 3.2.1.17/12650-88-3/4403-M |
| chicken egg white lysozyme. | |
| Lysozyme from hen egg white | 3.2.1.17/NA/10837059001 |

Alginate is broken by alginate lyases (EC 4.2.2.3), which are also called poly(beta-D-mannuronate) lyase, poly(beta-D-1,4-mannuronide) lyase, alginate lyase I, alginate lyase, alginase I, alginase II, and alginase. This enzyme catalyzes the eliminative cleavage of polysaccharides containing beta-D-mannuronate residues to give oligosaccharides with 4-deoxy-alpha-L-erythro-hex-4-enopyranuronosyl groups at their ends. As non-limiting examples, we list below alginate lyases available from Sigma-Aldrich®:

| Description | EC/CAS/Sigma |
|---|---|
| (Details on activity) | Aldrich no. |
| Alginate Lyase | 4.2.2.3/9024-15-1/A1603 |

As noted above, in the method of the invention, the polysaccharide is contacted with both the hydrolase and water. However, the contact step a) is carried out in the absence of solvent and therefore results in the formation of a solid reaction mixture.

Herein, a solvent is a liquid that forms a liquid phase in which a solute is dissolved (resulting in a solution) or that forms a continuous liquid matrix in which particles are dispersed/suspended (resulting in a dispersion or suspension) or are simply present (resulting in a slurry).

In the present invention, the water in the reaction mixture is a reactant in the desired hydrolysis reaction. However, even if the solid reaction mixture comprises some water for the hydrolysis reaction, it does not contain enough water for that water to act as a solvent. In other words, there is not enough water to surround a solute and dissolve it in a liquid phase or to form a continuous phase around particles (thus forming a dispersion, suspension, or slurry). In fact, there is no liquid phase in the solid reaction mixture. Rather, the solid reaction mixture has the appearance of and behaves as a solid. In particular, the reaction mixture is not free-flowing, it does not flow like a liquid. In fact, it is solid in appearance, presenting itself as a powder that is slightly humid (in embodiments sticky) to the touch. For certainty, the solid reaction mixture is not a slurry, in which a solid is mixed with a liquid forming a liquid or semi-liquid flowing mixture. The solid reaction mixture is not a dispersion, suspension or colloid, in which particles of a solid are dispersed or suspended in a liquid. The solid reaction mixture is not a solution in which a solute is dissolved in a liquid.

In embodiments, the ratio of the volume of liquid (in μL) to total solid weight (in mg) in the reaction mixture (ratio η) is at least 0.01 and at most about 3 μL/mg, preferably at least 0.01 and at most about 1.75 μL/mg. In preferred embodiments, then ratio is:
about 0.01, about 0.05, about 0.1, about 0.15, about 0.2, about 0.25, about 0.3, about 0.35, about 0.4, or about 0.45, about 0.5, about 0.55, about 0.6, about 0.65, about 0.7, about 0.75, about 0.8, about 0.85, about 0.9, about 0.95, about 1, about 1.05, about 1.1, about 1.15, or about 1.2 μL/mg or more, and/or
about 1.75, about 1.6, about 1.5, about 1.45, about 1.4, about 1.35, about 1.3, about 1.25, about 1.2, about 1.15, about 1.1, about 1.05, about 1, about 0.95, about 0.9, about 0.85, about 0.8, about 0.75, about 0.7, about 0.65, about 0.6, about 0.55, about 0.5, about 0.45, about 0.4, about 0.35 or about 0.3 μL/mg or less.

In more preferred embodiments, then ratio is between about 0.1 to about 1.5 μL/mg, between about 0.25 and about 1.75 μL/mg, between about 0.6 and about 1.6 μL/mg.

When the polysaccharide is a cellulose, the solid reaction mixture has preferably a ratio η of liquid volume, in μL, to total solid weight, in mg, between about 0.01 and about 3 μL/mg, preferably between about 0.01 and about 1.75 μL/mg, more preferably between 0.1 to about 1.5 μL/mg, yet more preferably between about 0.5 and about 1.5 μL/mg, even more preferably between about 0.75 and about 1.25 μL/mg, yet more preferably between about 0.9 and about 1.1 μL/mg, and most preferably is preferably about 1 μL/mg.

When the polysaccharide is a hemicellulose, the solid reaction mixture has a ration of liquid volume, in μL, to total solid weight, in mg, between about 0.01 and about 3 μL/mg, preferably between about 0.01 and about 1.75 μL/mg, more preferably between 0.1 to about 1.5 μL/mg, yet more preferably between about 0.25 and about 1.25 μL/mg, even more preferable between about 0.4 and about 1 μL/mg, yet more preferably between about 0.5 and about 0.7 μL/mg, and most preferably is preferably about 0.6 μL/mg.

When the polysaccharide is chitin, the solid reaction mixture has a ratio η of liquid volume, in μL, to total solid weight, in mg, between about 0.01 and about 3 μL/mg, preferably between about 0.01 and about 1.75 μL/mg, more preferably between 0.1 to about 1.75 μL/mg, yet more preferably between about 0.5 and about 1.75 μL/mg, even more preferable between about 1 and about 1.75 μL/mg, yet more preferably between about 1.5 and about 1.75 μL/mg, and most preferably is preferably about 1.6 μL/mg.

For comparison, a slurry can generally be defined as having a η ratio of at least about 2 μL/mg and suspensions/dispersions have even higher η ratios.

The quantity of water present in the reaction mixture can also be expressed as a function of the stoichiometric quantity of water necessary to achieve a complete hydrolysis of the polysaccharide. Defining the volume of the stoichiometric amount of water necessary to achieve a complete hydrolysis of the polysaccharide as “V”, in embodiments, the reaction mixture comprises between about 1V and about 20V of water, with the proviso that the ratio η must not exceed out 1.5 μL/mg. In preferred embodiments, the reaction mixture comprises
about 1V, about 2V, about 3V, about 4V, about 5V, about 6V, about 7V, about 8V, about 9V, or about 10V or more of water and/or
about 20V, about 19V, about 18V, about 17V, about 16V, about 15V, about 14V, about 13V, about 12V, aboug 11V, or about 10V or less of water.

In preferred embodiments, the reaction mixture comprises between 5V and about 15V, preferably about 8V to about 12V, and most preferably about 10V of water. Indeed, in preferred embodiments, especially those where the feedstock is cellulose, the mixture comprises about 10V of water, which appears to be optimum in such circumstances, in particular with the enzymes/feedstocks tested below. Indeed, at higher water volumes, enzymatic activity can be reduced (especially, when water rather than a buffer is used).

The volume of water can also be expressed using both of the above measurements. In embodiments, the volume of water present in the reaction mixture is between the volume of the stoichiometric amount of water necessary to achieve a complete hydrolysis of the polysaccharide (1V) and the volume of water yielding a ratio η of about 1 μL/mg.

The water present in the reaction mixture may be provided in the form of pure water (i.e. by itself rather than mixed with something else) or in the form of an aqueous buffer. Such buffer, if used, should preferably be selected according to the nature of the hydrolase to be used. Indeed, each enzyme has a well-known pH domain of stability and it is well within the skills of a person skilled in the art to select an appropriate buffer for a given enzyme. For example, the buffer can be a 2-(N-morpholino)ethanesulfonic acid (MES), 2,2-Bis(hydroxymethyl)-2,2′,2″-nitrilotriethanol (BIS-TRIS), N-(2-Acetamido)iminodiacetic acid (ADA), N-(2-Acetamido)-2-aminoethanesulfonic acid (ACES), 1,4-Piperazinediethanesulfonic acid (PIPES), β-Hydroxy-4-morpholinepropanesulfonic acid (MOPSO), 1,3-Bis[tris(hydroxymethyl)methylamino]propane (BIS-TRIS propane), N,N-Bis(2-hydroxyethyl)-2-aminoethanesulfonic acid (BES), 3-(N-Morpholino)propanesulfonic acid (MOPS), 2-[(2-Hydroxy-1,1-bis(hydroxymethy)ethyl)amino]ethanesulfonic acid (TES), 4-(2-Hydroxyethyl)piperazine-1-ethanesulfonic acid (HEPES), 3-(N,N-Bis[2-hydroxyethyl]amino)-2-hydroxypropanesulfonic acid (DIPSO), 4-(N-Morpholino)butanesulfonic acid (MOBS), 2-Hydroxy-3-[tris(hydroxymethyl)methylamino]-1-propanesulfonic acid (TAPSO), 2-Amino-2-(hydroxymethyl)-1,3-propanediol (TRIZMA® base), 4-(2-Hydroxyethyl)piperazine-1-(2-hydroxypropanesulfonic acid) Hydrate (HEPPSO hydrate), Piperazine-1,4-bis(2-hydroxypropanesulfonic acid) dihydrate (POPSO hydrate), 4-(2-Hydroxyethyl)-1-piperazinepropanesulfonic acid (EPPS), N-[Tris(hydroxymethyl)methyl]glycine (tricine), Diglycine (Gly-Gly), Diglycine (Bicine), N-(2-Hydroxyethyl)piperazine-N′-(4-butanesulfonic acid) (HEPBS), N-[Tris(hydroxymethyl)methyl]-3-aminopropanesulfonic acid (TAPS), 2-Amino-2-methyl-1,3-propanediol (AMPD), N-tris(Hydroxymethyl)methyl-4-aminobutanesulfonic acid (TABS), N-(1,1-Dimethyl-2-hydroxyethyl)-3-amino-2-hydroxypropanesulfonic acid (AM PSO), 2-(Cyclohexylamino)ethanesulfonic acid (CHES), 3-(Cyclohexylamino)-2-hydroxy-1-propanesulfonic acid (CAPSO), 2-Amino-2-methyl-1-propanol (AMP), 3-(Cyclohexylamino)-1-propanesulfonic acid (CAPS), 4-(Cyclohexylamino)-1-butanesulfonic acid (CABS), TAE (Tris base, acetic acid and EDTA), tris(hydroxymethyl)aminomethane (Tris)-HCl or potassium or sodium acetate, citrate, phosphate, or tartrate, or other type of buffers. In preferred embodiments, the buffer is a 2-(N-morpholino)ethanesulfonic acid (MES), tris(hydroxymethyl)aminomethane (Tris)-HCl, or a sodium acetate, citrate, phosphate or tartrate buffer. In more preferred embodiments, the buffer is a sodium acetate buffer. In preferred embodiments, the buffer has a pH ranging from about 3 to about 7, preferably from 4.5 to about 7, more preferably from about 5 to about 7, and most preferably of about 5.

As noted below, the water (pure or as a buffer) can be added to the reaction mixture by itself or it might be mixed with the hydrolase prior to being added to the reaction mixture.

The hydrolase concentration in the reaction mixture will depend on the nature of the polysaccharide feedstock, the nature and origin of the hydrolase itself, the level of activity of the hydrolase towards the polysaccharide feedstock, and the specific reaction conditions. In embodiments, the reaction mixture has a hydrolase concentration of about 0.01% to about 50% (expressed as w/w % based on the weight of the polysaccharide). In embodiments, the hydrolase concentration is:
about 0.01%, about 0.05%, about 0.1%, about 0.2%, about 0.25%, about 0.3%, about 0.4%, about 0.5%, about 0.6%, about 0.7%, about 0.75%, about 0.8%, about 0.9%, about 1%, about 1.25%, about 1.5%, about 1.75%, about 2%, about 3%, about 4%, about 5%, about 6%, about 7%, about 8%, about 9%, about 10% or more, and/or
about 50%, about 45%, about 40%, about 35%, about 30%, about 25%, about 20%, about 15%, about 14%, about 13%, about 12%, about 11%, about 10%, about 9%, about 8%, about 7%, about 6%, about 5%, about 4%, about 3%, about 2%, about 1.5%, about 1%, about 0.5% or less.

In preferred embodiments, the ratio is between about 0.05% and about 20%, more preferably between about 0.05% and about 5%, yet more preferably between about 0.25% and about 1.5%, even more preferably between about 0.5% and about 1.5%, and most preferably between about 1% and about 1.5%.

In the method of the invention, the hydrolase can be added to the polysaccharide in dry form (typically a powder, such as a lyophilized powder) or liquid form (i.e. dissolved in (at least part of) the water or the aqueous buffer as defined above). Both forms are commercially available, with the powder form being more prevalent. Alternatively, the hydrolase in liquid form can be prepared by dissolving a solid commercially preparation in water (or a buffer as described above).

Of note, in some cases, higher conversion rates may be obtained when the hydrolase is added to the reaction mixture in liquid form, preferably those prepared by dissolving a solid commercially preparation.

It should be noted that enzyme preparations in both forms, in particular commercial preparations, generally do not consist of pure hydrolase. Rather, they further comprise adjuvants such as culture medium components, buffer salts and/or other species. For example, the commercial powder preparations tested in some of the examples below contained between about 2 to about 30% hydrolase. Therefore, to achieve a given hydrolase concentration in the reaction mixture from a given enzyme preparation in powder or liquid form, in particular a commercial enzyme preparation, one should calculate the weight of powder, or the volume of liquid, to be used from the hydrolase concentration desired in preparation. When needed, the hydrolase concentration of a given enzyme preparation can be measured using standard procedures, such as the well-known Bradford assay (a colorimetric protein assay based on an absorbance shift of the dye Coomassie Brilliant Blue G-250).

Generally, increasing the concentration of hydrolase in the reaction mixture will increase the conversion rate observed for the saccharification reaction. However, in some cases, the use of large amounts of commercial enzyme preparations in powder form may be disadvantageous. Indeed, the use of a large amount of a commercial powder would lead to the addition of a large amount of adjuvants into the reaction mixture. In some cases, these adjuvants, when present in large amounts, may be deleterious to the saccharification reaction.

Somewhat similarly, in some cases, the use of large amounts of commercial enzyme preparations in solution form may be precluded. Indeed, the use of a large amount of a commercial solution would lead to the addition of a large quantity of water into the reaction mixture. However, as noted above, there is a maximum amount of water that can be added to the reaction mixture.

Therefore, it is preferable, when possible, to use enzyme preparations (either liquid of solid form) with high hydrolase concentrations, which means that they can be used in smaller amounts. However, these concentrated preparations may be more expensive than preparations with lower hydrolase concentrations. Further, the maximum hydrolase concentration of a given liquid enzyme preparation is capped and depend on the solubility of the enzyme in the solvent (water or buffer).

In embodiments, the hydrolase is (partly or entirely) added to the polysaccharide in dry form (typically as a powder). In such cases, the water (or aqueous buffer) is added to the polysaccharide separately from the hydrolase either before or after the hydrolase, preferably after the hydrolase. In more specific embodiments, the polysaccharide and hydrolase are first contacted together, mixed together or not (preferably mixed together), before the water (or aqueous buffer) is added. The mixing of these two solids (polysaccharide and enzyme) can be carried out manually or using a vortexer, a drum tumbler, a shaker mill, a planetary mill, an attritor, a mortar mill, an egg beater or any mechanical device that will allow the homogenization of the powders without denaturing the enzymes. The purpose of this mixing is simply to homogenize the solid mixture, not to impart energy or heat the solids. Care should be taken to avoid deactivating the hydrolase. Thus the mixing intensity and duration should be chosen accordingly. For example, in specific examples below, 200 mg samples were mixed manually for 10 seconds. In further embodiments, in addition to the hydrolase in solid form, hydrolase in liquid form is also added to the reaction mixture.

In embodiments, the hydrolase is (partly or entirely) dissolved in the water (or aqueous buffer as defined above) and then added to the polysaccharide. Such embodiments generally yield higher conversion rates. Indeed, it has been observed that the use of a concentrated solution of hydrolase is favorable to the reaction. If such addition does not provide all the water desired to the reaction mixture, then additional water (or aqueous buffer) can be further added to the reaction mixture. The hydrolase solution may be prepared, for example, by suspending 1 to 100 mg of a commercial hydrolase preparation in 1 mL liquid (water or buffer), which may yield for example a solution with a hydrolase concentration ranging from about 0.02 to about 30 mg/mL (in the case of some of the commercial preparation tested below).

In embodiments, the reaction mixture may further comprise one of more additives. These additives may be solid or liquid. Non-limiting examples of solid additives include powdered salts, metal or alkaline or alkaline earth oxides, silica beads or powder, alumina, polymer beads or abrasive powders. In the case of liquid additives, the volume added should be controlled so that the ration of the volume of liquid (in μL) in the reaction mixture to total solid weight (in mg) of the reaction mixture is at most about 1.5 μL/mg. Non-limiting examples of liquid additives include organic liquids, including ethylene glycol, glycerol, isopropanol, polyethylene glycol of any type or length, a detergent or a polymer such as poly (sorbitol methacrylate) or others.

### Step b)

The method of the invention then comprises b) the step of:
b)-i. mixing and then incubating the solid reaction mixture,
b)-ii. milling the solid reaction mixture, or
b)-iii. milling and then incubating the solid reaction mixture.

As will be explained in greater details below, during step b) (i, ii, or iii), the hydrolase effects the desired saccharification, which produces monosaccharides and/or oligosaccharides.

In embodiments, after step b)-i. or after step b)-iii., preferably after step b)-iii., the method further comprises:
the step c) of milling the solid reaction mixture [wherein step c) is carried out in the same way as step b)-ii. described herein] or
preferably the step c′) of milling and then incubating the solid reaction mixture [wherein step c′) is carried out in the same way as step b)-ii. described herein].

In further preferred embodiments, step c′) is repeated one or more times. As reported in the Examples below, steps c) and c′), particularly when repeated, allow reaching greater conversion rates.

It has indeed been surprisingly observed that the hydrolase is active and catalyses the hydrolysis of the polysaccharide in the solid reaction mixture. Traditionally, the use of enzymes has been restricted to their natural, aqueous reaction media. Water is still the solvent of choice when using enzymes—see for example US 2016/0002689 and US 2016/0032339. The switch to other solvents, in particular organic solvents, and other reaction media seemed impossible at first in light of the idea that enzymes (and other proteins) are denatured, i.e. lose their native structure and thus catalytic activity, in such reaction media. Some enzyme-catalysed reactions have been successfully carried out in organic solvents, and even in supercritical fluids and the gas phase, specifically with crystalline enzymes and enzymes lyophilized under specific conditions. Nevertheless, their very limited range of stability with respect to temperature, solvents, pH value, ionic strength, and salt type remains a decisive weakness of enzymes—see Klibanov, Nature, 2001, 409, 241 and Bommarius, Annu. Rev. Biomol. Eng. 2015, 6, 319. Furthermore, in all cases, there is always a fluid solubilizing the enzyme, allowing it to fold correctly and allowing it to contact its intended substrate. The use of enzymes, especially conventional wild-type enzymes, in the solid state, i.e. the ability of the hydrolases to operate without a solvent or fluid medium, in the present invention was highly unexpected. To the inventor's knowledge, enzyme activity in the solid state, particularly in the absence of a solvent and using wild-type enzymes, has never previously been suggested or demonstrated for non-immobilized enzymes.

It has also been surprisingly observed that milling of the solid reaction mixture does not deactivate the hydrolase. This is quite unexpected because enzymes are known to be sensitive to various stresses including high and low temperatures, ionic strength, chaotropic salts, organic solvents, denaturants, and gas-liquid and solid-liquid interfaces—see Bommarius, Annu. Rev. Biomol. Eng. 2015, 6, 319. This means that a mechanochemical approach to using enzymes (i.e. milling enzymes in a solid reaction mixture) would have been expected to fail and to inactivate the enzyme because of the presence of mechanical stress, potential local heating (“hot spots”), and the presence of a solid-liquid interface. The secondary and tertiary structures of the enzymes, which govern their activity, would have been expected to change when exposing the enzymes to mechanical energy. The ability of enzyme to survive mechanical processing, or mechanical activation in a solvent-free environment was therefore quite unexpected.

It has also been surprisingly found that milling not only speeds up hydrolysis, but, in fact, when used before incubation, allows reaching conversion rates that are superior to those obtained with incubation only. In other words, milling prior to incubating allows surpassing the plateau conversion levels observed with incubation alone.

Step b)-i.

In step b)-i., the solid reaction mixture is first mixed and then incubated.

The mixing of the reaction mixture can be carried out manually or using any suitable mixing means known to the skilled person. Indeed, the purpose of this mixing is simply to homogenize the solid mixture, not to impart energy or heat to the mixture. Care should be taken to avoid deactivating the hydrolase during this mixing. Thus, the mixing intensity and duration should be chosen accordingly. Non-limiting examples of mixing means includes a vortexer, a drum tumbler or any other mechanical device that will allow the homogenization of the powders without denaturing the enzymes. For example, in specific examples below, 200 mg samples were mixed manually for 30 seconds and 10 mg samples were vortexed for 5 seconds.

Then, the mixture is incubated. Herein, incubating means keeping the reaction mixture in conditions (temperature, relative humidity, etc.) that allow, and preferably favor, the hydrolysis of the polysaccharide feedstock by the hydrolase. These conditions will depend on the nature of the polysaccharide feedstock and of the hydrolase. Preferably, the mixture is incubated in conditions allowing maximum enzymatic activity, which conditions are typically known to the skilled person. In embodiments, the mixture is incubated at a temperature from about 0° C. to about 80° C., preferably from about 20° C. to about 60° C., more preferably from about 30° C. to about 55° C., yet more preferably from about 40° C. to about 50° C., and most preferably about 45° C. In embodiments, the mixture is incubated under a relative humidity ranging from normal atmospheric conditions to 100% relative humidity, preferably from about 50% to about 100% relative humidity, more preferably from about 75% to about 100% relative humidity, yet more preferably from about 90% to about 100% relative humidity, and more preferably of about 100% relative humidity.

The length of the incubation will depend on the conversion rate desired. Longer incubation times tend to lead to higher conversion rates. The length of the incubation will also depend whether steps c) or c′) will be carried and whether and how many times step c′) will be repeated. Generally, the incubation may last between about 30 minutes and about 30 days. Preferably, the incubation lasts:
about 30 mins, about 45 minutes, about 1 h, about 4 h, about 8 h, about 12 h, about 16 h, about 20 h, about 1 day, about 2 days, about 3 days, about 4 days, about 5 days, about 6 days, about 7 days, about 8 days, about 9 days, about 10 days, about 15 days, about 20 days or about 25 days or more, and/or
about 20 days, about 15 days, about 14 days, about 13 days, about 12 days, about 11 days, about 10 days, about 9 days, about 8 days, about 7 days, about 6 days, about 5 days, about 4 days, about 3 days, about 2 day, about 1 day, about 20 h, about 16 h, about 12 h about 8 h, about 4 h, about 1 h or less.

Most preferably, the incubation lasts between about 1 hour and 7 days. In embodiments where step c′ is carried, and optionally repeated one or more times, the incubation time is typically kept for each milling/incubation cycle on the lower end of the incubation time ranges provided above. In such embodiments, the incubation time is preferably about 1 hour to 1 day for each cycle.

During incubation, in some circumstances, the conversion (hydrolysis) may first progress relatively rapidly and then slow down and even tend to plateau. There is little benefit to incubating the mixture once a plateau has been reached. Therefore, incubation is advantageously stopped once the hydrolysis has slowed down to a point at which additional incubating time is unadvantageous. Step c′, especially when repeated, has been surprisingly shown below to help overcome such plateaus and allow reaching higher conversion rates.

Step b)-ii.

In step b)-ii., the solid reaction mixture is milled.

The purpose of this milling is speed up hydrolysis by imparting mechanical energy to the reaction mixture. Nevertheless, care should be taken to avoid deactivating the hydrolase. The milling can be carried out using a ball mill (including shaker, planetary, attrition, magnetic, and tumbler mills), a roller mill, a knife mill, a mixer mill, a disk mill, a cutting mill, a rotor mill, a pestle mill, a mortar mill, or a kneading trough, preferably a ball mill, more preferably a shaker mill. Depending on the type of mill used, the milling can last from about 5 to about 90 minutes. In preferred embodiments, the milling lasts:
about 5, about 10, about 15, about 20, about 25, about 30, about 35, about 40, about 45, about 50, about 55, or about 60 minutes or more, and/or
about 90, about 75, about 60, about 55, about 45, about 40, about 35, about 30, about 25, about 20, about 15, or about 10 minutes or less.

In preferred embodiments, the milling lasts from about 5 to about 60 minutes, more preferably from about 15 to about 60 min, and most preferably from about 30 to about 60 mins.

In embodiments, the mill is set at a frequency ranging from about 0.5 to about 100 Hz, with preferred frequency ranges depending on the type of mill used. For example, for a planetary mill, the frequency is preferably from about 3 to about 10 Hz. For a shaker mill, the frequency is preferably from about 20 to about 40 Hz, more preferably from about 25 to about 35 Hz and is most preferably about 30 Hz. For a mixer mill, the frequency is preferably from about 60 to about 80 Hz.

The milling container and impact agent are chosen in the purpose of conveying energy to the reactional system without inactivating the enzyme. Non-limiting example of suitable materials include plastic (PM MA), stainless steel, Teflon, zirconia, agate, and tungsten carbide—see the Examples below. In ball mills, the impact agents are balls, and their shape and nature may vary depending on the chosen milling mode. Their material, size, weight and number are determined according to the size and shape of the milling vessel as well as sample volume. Impact agents of different sizes may be used simultaneously.

Such milling has relatively low energy requirements. Further, it is a soft mild grinding, but it has nevertheless, been shown below to be sufficient to provide the unexpected results reported herein. Generally, this mild milling produces little increase in temperature of the reaction mixture. Temperature elevation may be observed but usually the temperature does not raise above about 80° C., preferably not above about 40° C. In embodiments, the milling temperature varies between about 0 to about 80° C., preferably between about 20 and about 40° C., and most preferably about room temperature.

Step b)-iii.

In step b)-iii., the solid reaction mixture is incubated after being milled. The milling in step b)-iii. is as described for step b)-ii above. The incubation in step b)-iii. is as described for step b)-i. above.

### Advantages and Potential Applications

In embodiments, the method of the invention may have one or more of the following advantages.

The invention is based on the use of non-immobilized enzymes under solvent-free conditions. In particular, we demonstrate below that enzymatic hydrolytic degradation of cellulose into smaller oligocelluloses and/or glucose, by cellulase, without solvents, at room temperature is possible and that mechanical milling can be conducted over extended periods of time without deactivating the enzyme.

The method of the invention thus avoids using solvents, minimizes water use/pollution, and enables the action of enzymes on poorly soluble solid substrates. This invention is advantageous over the existing processes for breakdown and exploitation of biopolymers, as it can operate on poorly soluble, non-reactive substrates without the need for dissolution, in that way avoiding solvents (water, ionic liquids).

The method of the invention represents a clean, inexpensive (using readily available and cheap wild type enzymes) and efficient route for the degradation of polysaccharides, which is a central problem of modern biowaste valorization, and a stumbling block in the use of biowaste as feedstocks for fuel, chemicals and in other related (e.g. pharmaceuticals) industries. So far, processing and breakdown of such polymers into simpler, useful constituents has been an arduous and often energy-consuming process that requires aggressive chemicals, such as strong acids (sulfuric, hydrochloric acids), bases (sodium hydroxide), transition metal salts (e.g. ammonia-copper(II) solution for cellulose dissolution), expensive chemicals (e.g. ionic liquids). The present invention avoids aggressive acidic, basic or transition metal reagents or organic solvents. Importantly, the invention is capable of conducting biopolymer hydrolysis reactions with no auxiliary materials (in that way being also advantageous over methods that utilize low-toxicity inorganic additives, such as zeolites, clays or diatomaceous earth, that require specialized separation techniques). As there are no additives, the separation of polymer breakdown products from the starting feedstock is simple and, in embodiments, based on washing only. The invention allows biopolymer breakdown with low energy input, by different combinations of short milling processes and/or low-temperature aging. The present invention provides an unprecedented clean route to degradation of such polymers.

Furthermore, the reaction is selective, the product(s) being dictated by the choice of enzyme. When using cellulose and cellulase, the products are oligosaccharides, glucose, or a mixture thereof.

It should be noted that steps a) and b) can advantageously be carried out in the absence of harsh or expensive reagents (strong acids or bases, transition metal salts, ionic liquids) and/or in mild conditions, i.e. under atmospheric pressure and at about room temperature (milling, mixing, incubating) or moderate temperatures (incubating).

Overall, the method of the invention is expected to be useful for the valorization of waste polysaccharides (e.g. cellulose in wood, corn, nuts, grass, paper, fabric; chitin in crab, lobster, shrimp shells; starch from different crops) and their use as feedstocks for renewable fuel (biofuels), chemical (pharmaceuticals) and polymer industry.

### Definitions

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Similarly, herein a general chemical structure with various substituents and various radicals enumerated for these substituents is intended to serve as a shorthand method of referring individually to each and every molecule obtained by the combination of any of the radicals for any of the substituents. Each individual molecule is incorporated into the specification as if it were individually recited herein. Further, all subsets of molecules within the general chemical structures are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. In embodiments, it may mean plus or minus 10% or plus or minus 5% of the numerical value qualified.

Unless otherwise defined, all technical and scientific terms used herein have the same meaning as commonly understood by one of ordinary skill in the art to which this invention belongs.

Other objects, advantages and features of the present invention will become more apparent upon reading of the following non-restrictive description of specific embodiments thereof, given by way of example only with reference to the accompanying drawings.

### DESCRIPTION OF ILLUSTRATIVE EMBODIMENTS

The present invention is illustrated in further details by the following non-limiting examples.

### EXAMPLE 1

### General Methods

Chemicals. Aspergillus niger (Product no. 22178) and Trichoderma reesei (Product no. C8546) enzymes were purchased from Sigma-Aldrich (Oakville and Milwaukee, respectively). Microcrystalline cellulose (MCC) was obtained from Sigma-Aldrich (Oakville). Sodium acetate buffer was prepared using solid sodium acetate from Sigma-Aldrich dissolved to a final concentration of 50 mM in deionized water; pH was later adjusted to 5.0 using 1N HCl.

Most commercially available cellulase enzymes are constituted of a mixture of several cellulases, which, combined together, display all three types of activities described in FIG. 1. This was the case of the lyophilized cellulase powders from A. niger and T. reesei used in this study. Although labelled as “enzyme”, the commercial preparations in fact comprised several other components, such as culture medium elements or buffer salts. The ratio of protein in this mixture was evaluated using the standard Bradford assay which revealed a proteinic content of 2% and 30% for the A. niger and T. reesei powders, respectively. Consideration of this enzyme concentration is necessary when comparing the enzymatic activity of the commercial preparations. The protein content comprised at least four enzymes of molecular weights between 25 and 50 kDa as revealed by gel chromatography analysis.

All experiments were done at least in triplicate and error bars represent standard deviation.

Glucose and Polysaccharides Quantification (DNS Protocol). All digestion reactions were monitored using 3,5-dinitrosalicylic acid (DNS) which reacts with the reducing end of sugars to afford 3-amino-5-nitrosalicylic acid which strongly absorbs at 540 nm. This method allows the non-discriminate detection of glucose and oligosaccharides.

Hydrolysis progress was determined by considering cellulose as a single long chain of glucose units and neglecting its two ends. The molecular weight of each cellulose repeat unit is that of sugar amputated from a water molecule (162.14 g/mol). For example, complete hydrolysis of a 10 mg/mL suspension of cellulose would therefore correspond to the release of 11 mg of glucose in the same volume corresponding to a concentration of 62 mM. All hydrolysis percentages reported in the present Examples were calculated as follow:

$$\frac{\begin{matrix}concentration of glucose and oligosaccharides \\ measured using 3 , 5 \text{-} dinitrosalicylic acid\end{matrix}}{\begin{matrix}theoritical concentration of glucose \\ produced by complete hydrolysis\end{matrix}} \times 100.$$

We note that cellulose crystals are not infinitely long; the average molecular weight of each unit in the polymer is therefore higher than the one we considered. As a consequence, the complete digestion of actual crystals would in fact lead to a slightly lesser final concentration of glucose than that calculated above, and the hydrolysis percentages values presented herein are most likely somewhat underestimated. Secondly, we also note that the DNS reagent reacts with a 1/1 stoichiometry with the reducing end of a polysaccharide of any length. The present protocol therefore quantifies glucose and polysaccharides in an identical manner. Thus, unless full digestion to glucose is achieved, it should be considered that the values reported in the present Examples somewhat underestimate the hydrolysis of crystalline cellulose.

DNS Reagent Preparation. 1 g of 3,5-Dinitrosalicylic acid (DNS) was suspended in about 50 mL deionized water. 30 g of sodium potassium tartrate tetrahydrate was added in small portions. 20 mL of NaOH (2M) was added and the volume adjusted to 100 mL with deionized water. The mixture was then filtered over cotton to afford the DNS reagent which was stored in an inactinic bottle at 4° C.

DNS Reagent Test. 200 μL of the desired sample were introduced in a 1.5 mL Eppendorf vial and mixed with 100 μL of the DNS reagent. The solution was vortexed for 2 s and incubated for 5 min in a boiling water bath. After cooling down to room temperature, 200 μL of the reacted sample was introduced in a well of a 96-well microtiter plate. Absorption at 540 nm was measured using a Spectramax i3x from Molecular Devices. The test was calibrated using freshly made glucose solutions of known concentrations. Linear regression afforded the correspondence equation between absorption and reducing end sugar concentration. In cases of high glucose content, the boiled samples were diluted by a factor 4 to allow accurate measurement of absorption.

### EXAMPLE 2

### Accelerated Aging (AA)

Kinetics of AA. 18 samples containing 10±0.05 mg microcrystalline cellulose (C) were prepared. MCC was put in contact with 5 μL of a freshly prepared solution of enzyme preparation from A. niger 10 mg/mL corresponding to a total hydrolase content of 0.01%. All samples were shaken manually for 10 s. The resulting mixture consisted of a sticky powder with no observable liquid phase. All samples were introduced simultaneously in an incubator set at 45° C. and 100% relative humidity (AA conditions). Samples were removed in triplicates at 0, 1, 2, 6, 9 and 19 days. The powder was suspended in deionized water so as to obtain a 10 mg/mL suspension of cellulose which was vortexed for 5 s and then centrifuged for 1 min at 17.9×1,000 g. The supernatant was analysed through the DNS protocol described in Example 1.

Results indicate that the reaction proceeded within 6 days to reach a plateau at 1.3% conversion (FIG. 2). No further evolution of the mixture was observed.

Control experiments revealed that the removal of any of the reaction components prevented the reaction from proceeding. Most surprising was the fact that the ambient humidity was not enough to initiate the reaction and an initial addition of liquid is necessary. The dissolution of the enzyme preparation prior to contact with cellulose afforded better yields than contact between the two solids followed by liquid addition.

### EXAMPLE 3

### AA of Preparations from Different Origins in Different Assisting Liquids

5 μL of a freshly prepared solution of A. niger or T. reesei commercial preparation at 100 mg/mL in water or acetate buffer was added to 10 mg of MCC; 5% loading w/w corresponding to an actual hydrolase content of 0.1 and 1.5% respectively. The samples were vortexed for 5 s using a VWR Mini-Vortexer set on level 10 and incubated in AA conditions for 7 days. The resulting solid was suspended in deionized water so as to obtain a 10 mg/mL suspension of cellulose which was vortexed for 5 s and then centrifuged for 1 min at 17.9×1,000 g. The supernatant was analysed through the DNS protocol described in Example 1.

Better conversion was obtained from the T. reesei preparation affording 17% conversion with minimal energy input. The reaction proceeds just as well in water as in buffer. Without being bound by theory, this could result from the direct reconstitution of the pre-lyophilization culture medium or buffer by addition of water. The use of buffer can actually be detrimental by increasing the salt concentration above the optimal conditions as seen with the T. reesei preparation in acetate buffer.

TABLE 1
Conversions reached in AA conditions depending on the
origine of the enzyme preparation and assisting liquid

Assisting liquid

| Enzyme origin | Water | Acetate buffer |
|---|---|---|
| Aspergillus niger | 4.8 ± 0.3 | 5.99 ± 0.3 |
| Trichoderma. reesei | 17.0 ± 2.4 | 12.5 ± 4.9 |

### EXAMPLE 4

### AA with Additives in the Assisting Liquid

5 μL of a freshly prepared solution of A. niger commercial preparation at 100 mg/mL in acetate buffer containing 2% or 5% glycerol or ethylene glycol was added to 10 mg of MCC; 5% loading w/w corresponding to an actual hydrolase content of 0.1%. The samples were vortexed for 5 s using a VWR Mini-Vortexer set on level 10 and incubated in AA conditions for 7 days. The resulting solid was suspended in deionized water so as to obtain a 10 mg/mL suspension of cellulose which was vortexed for 5 s and then centrifuged for 1 min at 17.9×1,000 g. The supernatant was analysed through the DNS protocol described in Example 1.

The addition of small percentage of diols or triols in the assisting liquid can increase the reaction yields by a factor of almost two.

TABLE 2
Influence of additives (nature and ratio)
on conversions reached in AA conditions

Additive concentration

| Additive | 2% | 5% |
|---|---|---|
| Ethylene glycol | 9.1 ± 0.1 | 8.1 ± 0.3 |
| Glycerol | 9.5 ± 0.2 | 7.5 ± 0.7 |

### EXAMPLE 5

### Influence of Assisting Liquid Volume in AA Reactions

5 μL of a freshly prepared solution of A. niger commercial preparation at 100 mg/mL in water or acetate buffer was added to 10 mg of MCC; 5% loading w/w corresponding to an actual hydrolase content of 0.1% followed immediately by the addition of water (0, 5, 15 or 45 μL). The samples were vortexed for 5 s using a VWR Mini-Vortexer set on level 10 and incubated in AA conditions for 7 days. The resulting solid was suspended in deionized water so as to obtain a 10 mg/mL suspension of cellulose which was vortexed for 5 s and then centrifuged for 1 min at 17.9×1,000 g. The supernatant was analysed through the DNS protocol described in Example 1.

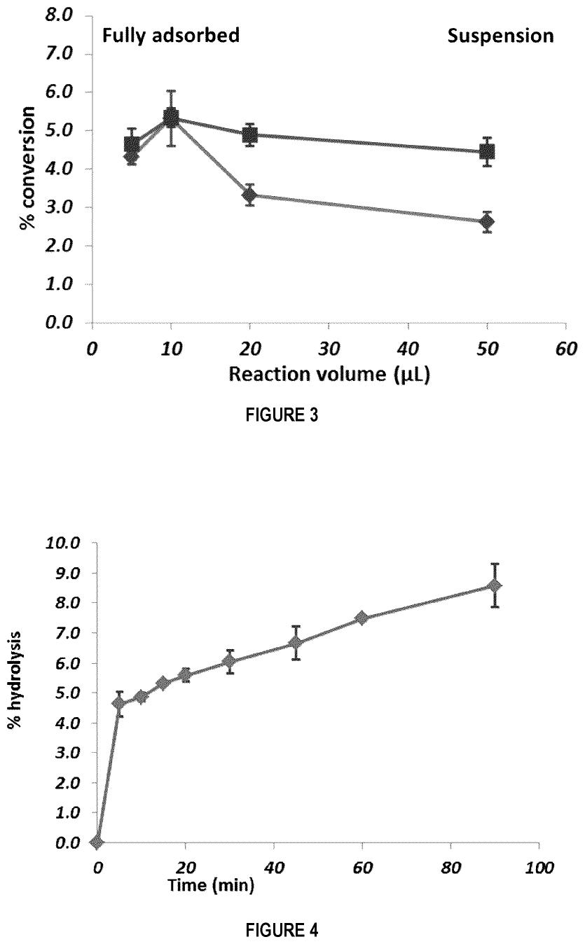

For a given quantity of enzyme preparation, maximum conversion was observed for a reaction volume of 10 μL (η=1) both in water and buffer (FIG. 3) although the maximum is less marked for buffer. This results indicates it is profitable to work with solids rather than slurries (V=20 μL, η=2) or colloidal suspension (V=50 μL, η=5).

### EXAMPLE 6

### Shaker Mill Reactions (SM)

In a typical experiment, 200 mg MCC were contacted with 10 mg enzyme preparation from A. niger or T. reesei, 5% w/w loading corresponding to a hydrolase ratio of 0.1 and 1.5% respectively. The powders were mixed and introduced in a plastic milling vessel (14 ml) containing 2 stainless steel balls 7 mm in diameter. 200 μL of water or acetate buffer were added and the mixture was milled in a shaker mill MM400, from Retsch or FTS 1000 from Form Tech Scientific for 60 min at 30 Hz. After milling, the resulting paste is harvested and suspended in deionized water so as to obtain a 10 mg/mL suspension of cellulose. The samples were boiled for 30 min to inactivate enzymes. A 1 mL aliquot of the suspension was vortexed for 5 s and then centrifuged for 1 min at 17.9×1,000 g. The supernatant was analysed through the DNS protocol described in Example 1.

Results show that 1 hr of milling leads to similar conversions as 1 week of incubation. This provides proof that the shaker mill is an adapted means to provide enzymes with the required energy to operate and that sustained milling does not lead to immediate nor fast denaturation of the enzymes. The present example actually provides proof that the enzymes remain active throughout the whole milling phase and after.

TABLE 3
Conversions reached in SM conditions depending on
the origin of the enzyme preparation and assisting liquid

Assisting liquid

| Enzyme origin | Water | Acetate buffer |
|---|---|---|
| Aspergillus niger | 3.4 ± 0.9 | 2.8 ± 0.7 |
| Trichoderma reesei | 11.1 ± 1.2 | 12.5 ± 4.9 |

### EXAMPLE 7

### Kinetics of SM Reactions

2 g MCC were contacted with 100 mg enzyme preparation from A. niger or T. reesei, 5% w/w loading corresponding to a hydrolase ratio of 0.1 and 1.5% respectively. The powders were mixed and introduced in a stainless steel milling vessel (volume 24 mL) containing 2 stainless steel balls 10 mm in diameter. 1 mL of water was added and the mixture was milled in a shaker mill from MM400 from Retsch for 90 min at 30 Hz. Aliquots of typically 20 to 30 mg were taken at 5, 10, 15, 20, 30, 45, 60 and 90 min. The collected samples were suspended in deionized water so as to obtain a 10 mg/mL suspension of cellulose which was boiled for 30 min to inactivate enzymes. The suspension was then vortexed for 5 s and centrifuged for 1 min at 17.9×1,000 g. The supernatant was analysed through the DNS protocol described in Example 1.

The conversion shows two phases with a remarkable 4.5% conversion within the first 5 min of the reaction followed by a linear increase over the next 85 min in the case of the T. reesei preparation (FIG. 4). A. niger presents the same profile with lower conversions in coherence with its lower hydrolase content. The transition between the two phases corresponds to the formation of a thick paste from the initial wet powder as a consequence of the loss of crystallinity due to both mechanical and enzymatic action. Once the paste is formed, the transmission of energy may be less efficient than during the powder phase, explaining the reduced rate.

### EXAMPLE 8

### Combining Milling and Accelerated Aging (SMAA)

In a typical experiment, 200 mg MCC were contacted with 10 mg enzyme preparation from A. niger or T. reesei, 5% w/w loading corresponding to a hydrolase ratio of 0.1 and 1.5% respectively. The powders were mixed and introduced in a plastic milling vessel (volume 14 mL) containing 2 stainless steel balls 7 mm in diameter. 200 μL of water or acetate buffer were added and the mixture was milled in a shaker mill MM400 from Retsch or FTS1000 from Form Tech Scientific for 60 min at 30 Hz. After milling, the resulting paste was harvested and incubated in AA conditions for 7 days. It was then suspended in deionized water so as to obtain a 10 mg/mL suspension of cellulose which was boiled for 30 min to inactivate enzymes. The suspension was then vortexed for 5 s and centrifuged for 1 min at 17.9×1,000 g. The supernatant was analysed through the DNS protocol described in Example 1.

Results indicate a factor 2 to 3 increase in conversion compared to both AA and SM reactions in similar conditions. It provides that the milling conditions do not lead to complete denaturation of the enzymes and that at least part of them operate during the subsequent aging phase. Surprisingly, this week-long activity is observed in the paste formed during the SM phase to the same or a better extent than in the wet powder of AA reactions. The SM phase also allows to overcome the plateau observed for AA reactions with an effect that can be cooperative and the resulting conversion from SMAA is superior to the sum of conversions observed for separate SM and AA reactions in similar conditions.

TABLE 4
Conversions reached in SMAA conditions depending on
the origin of the enzyme preparation and assisting liquid

Assisting liquid

| Enzyme origin | Water | Acetate buffer |
|---|---|---|
| Aspergillus niger | 8.0 ± 1.1 | 9.3 ± 1.3 |
| Trichoderma reesei | 23.9 ± 0.6 | 18.6 ± 1.9 |

### EXAMPLE 9

### Influence of Preparation Loading in SM and SMAA Reactions

200 mg MCC were contacted with 25, 50 or 100 mg enzyme preparation from A. niger, 12.5, 25 and 50% w/w loading corresponding to a hydrolase ratio of 0.25, 0.5 and 1%. The powders were mixed and introduced in a plastic milling vessel (volume 14 mL) containing 2 stainless steel balls 7 mm in diameter. Water or acetate buffer (100 μL) was added and the mixture was milled in a shaker mill MM400 from Retsch or FTS1000 from Form Tech Scientific for 30 min at 30 Hz. After milling, the resulting mixture was partitioned. Roughly half (samples labelled “SM” below) was suspended in deionized water so as to obtain a 10 mg/mL suspension of cellulose which was boiled for 30 min to inactivate enzymes. The suspension was then vortexed for 5 s and centrifuged for 1 min at 17.9×1,000 g. The supernatant was analysed through the DNS protocol described in Example 1. The other half was incubated in AA conditions for 7 days then suspended and analyzed following the same protocol (samples labelled “SMAA” below).

Increasing the preparation loading did not result in any observable conversion during milling while incubating the milled material allowed to reach up to 24% conversion. This shows that the above SMAA process, where water is simply added to a cellulose/enzyme mixture, quite unexpectedly allows bypassing the maximum enzyme loading limits imposed by the addition of the enzyme to the reaction mixture as a solution.

TABLE 5
Influence of hydrolase ratio
on the conversions of SM and SMAA reactions

| Hydrolase | SM | | SMAA | |
|---|---|---|---|---|
| ratio | Water | Acetate Buffer | Water | Acetate Buffer |
| 0.25% | 1.0 ± 0.5 | 0.6 ± 0.8 | 7.4 ± 1.6 | 5.1 ± 1.5 |
| 0.5% | 2.5 ± 0.5 | −0.7 ± 0.7 | 17.1 ± 1.9 | 9.8 ± 2.6 |
| 1% | −1.4 ± 1.0 | −0.8 ± 1.9 | 24.0 ± 4.5 | 22.7 ± 1.5 |

### EXAMPLE 10

### Influence of Ball Material in SM and SMAA Reactions

200 mg MCC were contacted with 10 mg enzyme preparation from A. niger, 5% w/w loading corresponding to a hydrolase ratio of 0.1%. The powders were mixed and introduced in a Teflon milling vessel (volume 24 mL) containing 2 balls 7 mm in diameter made of tungsten carbide, stainless steel, zirconia, or agate (in order of decreasing density) or no balls at all. Water (200 μL) was added and the mixture was milled in a shaker mill MM400 from Retsch for 60 min at 30 Hz. After milling, the resulting mixture was partitioned. Roughly half (samples labelled “SM” below) was suspended in deionized water so as to obtain a 10 mg/mL suspension of cellulose which was boiled for 30 min to inactivate enzymes. The suspension was then vortexed for 5 s and centrifuged for 1 min at 17.9×1,000 g. The supernatant was analysed through the DNS protocol described in Example 1. The other half (samples labelled “SMAA” below was incubated in AA conditions for 7 days then suspended and analyzed following the same protocol.

While the nature of the impact agent did not influence much the SM reactions, a clear tendency can be observed in SMAA favoring heavier balls. This shows that enzymes can survive high energy impacts in the shaker mill and that the resulting material is more favorable to AA. Interestingly, the absence of impact agents led to conversions comparable to those of AA reactions presented in Example 3 which further demonstrates the influence of the milling phase.

TABLE 6
Influence of ball material on SM and SMAA reactions outcome

| Ball material | SM | SMAA |
|---|---|---|
| Tungsten carbide | 5.0 ± 0.4 | 13.4 ± 0.8 |
| Stainless steel | 3.2 ± 0.4 | 11.7 ± 0.7 |
| Agate | 2.7 ± 0.6 | 8.5 ± 1.5 |
| Zirconia | 2.5 ± 0.8 | 9.3 ± 1.5 |
| No balls | 1.3 ± 0.1 | 5.7 ± 0.3 |

### EXAMPLE 11

### Successive SMAA Reactions (SMAA)ₙ

2 g MCC were contacted with 100 mg enzyme preparation from A. niger, 5% w/w loading corresponding to a hydrolase ratio of 0.1%. The powders were mixed and introduced in a Teflon milling vessel (25 ml volume) containing 2 stainless steel balls 10 mm in diameter. Water (2 mL) was added and the mixture was milled in a shaker mill MM400 from Retsch for 5 min at 30 Hz. After milling, an aliquot (˜20 mg) was collected and frozen. The remaining paste was incubated in AA conditions for 23 h after which the milling phase was repeated in identical conditions. The operation was repeated every day for 3 weeks. All aliquots were then suspended in deionized water so as to obtain 10 mg/mL suspensions of cellulose and boiled for 30 min to inactivate enzymes. The suspension was centrifuged for 1 min at 17.9×1,000 g. The supernatant was analysed through the DNS protocol described in Example 1.

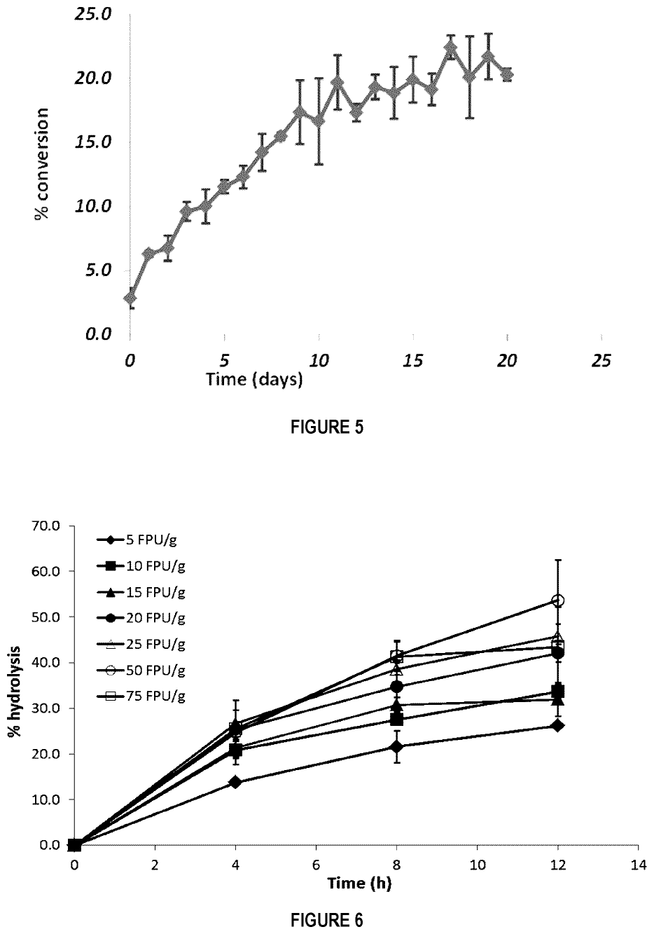

Daily milling of the reaction mixture shows that it is possible to reactivate the enzymes for up to 2 weeks (FIG. 5) to reach conversions of 20%, more than twice the ones obtained after just one milling phase, using only a 0.1% hydrolase content and minimal energy input. The plateauing of SMAA reactions described in previous examples is therefore linked to a local lack of substrate rather than enzyme deactivation. It is believed that the successive milling phases allow the renewal of the enzyme immediate environment and further digestion of cellulose.

### EXAMPLE 12

### Hydrolysis of Cellulose (MCC) with T. longibrachiatum Cellulases

We have found that a commercial T. longibrachiatum cellulases preparation (“food grade” purchased from CREATIVE Enzymes) was superior to the above commercial T. reesei cellulase (and much more active than the above commercial A. niger cellulase), for the hydrolysis of microcrystalline cellulose (MCC, obtained from Sigma-Aldrich (Oakville)), even when adjusting to the same protein content. Hence, unless noted otherwise, all the results below are for a commercial T. longibrachiatum cellulases preparation, sold as a lyophilized powder, which was found to have a protein content of 12% (by Bradford assay).

Also, unless otherwise noted, all experiments with cellulose consist of a successive SMAA reactions regime [(SMAA)ₙ also sometimes called RAging herein] of 5 min milling (30 Hz, r.t.) and 55 min aging (55° C.) repeated over 12 cycles.

The table below compares the yield (% hydrolysis) obtained for the conversion of MCC to glucose using T. longibrachiatum and T. reesei in various conditions.

| | Enzyme Loading | η | Aging Temp. | Hydrolysis |
|---|---|---|---|---|
| Enzyme | (FPU/g cellulose)* | (μL/mg) | (° C.) | (%) |
| T. reesei | 16 | 0.5 | 45 | 19.6 |
| T. reesei | 16 | 0.5 | 60 | 24.7 ± 1.6 |
| T. reesei | 80 | 0.4 | 60 | 25.8 |
| T. longi. | 25 | 0.4 | 60 | 35.0 |
| T. longi. | 25 | 0.4 | 60 | 34.9 |
| T. longi. | 25 | 0.8 | 60 | 49.2 ± 2.2 |
| T. longi. | 25 | 0.8 | 60 | 50.2 |

*FPU refers to “filter-paper units” calculated as per IUPAC guidelines - see Ghose, T. K. 1987. “Measurement of Cellulase Activities.” Pure & Appl. Chem. 59: 257-268.

Increasing the amount of protein (T. longibrachiatum enzyme, with a constant 200 μL of water and 200 mg MCC) used from 0.6% to 3% (corresponding to 5-25 FPU/g MCC) led to an increased yield of glucose, however increasing the enzyme loading beyond that did not translate into a further increase in yield—see FIG. 6. Based on this result, all experiments discussed below were performed with 3% (w/w) protein/MCC.

NB. As noted above, we used commercial preparations which contained 100% protein, i.e. the preparations contained adjuvants. Herein and in the following examples, when we provide a value as a weight (mg) of enzyme (mostly in plots), we refer to the total weight of the commercial enzyme preparation as purchased (including adjuvants). This number is somewhat misleading however because the commercial enzyme preparations contain 100% protein (enzyme). This is why, herein and in the following examples, we express the amount of enzyme used as a percentage of the weight of protein used (not the total weight of the commercial preparation) over the weight of substrate.

The amount of water was varied and found to be optimal at η˜1 for hydrolysis of MCC with cellulases. Hence, unless otherwise noted, all experiments discussed in the present Example below were performed with η=1.

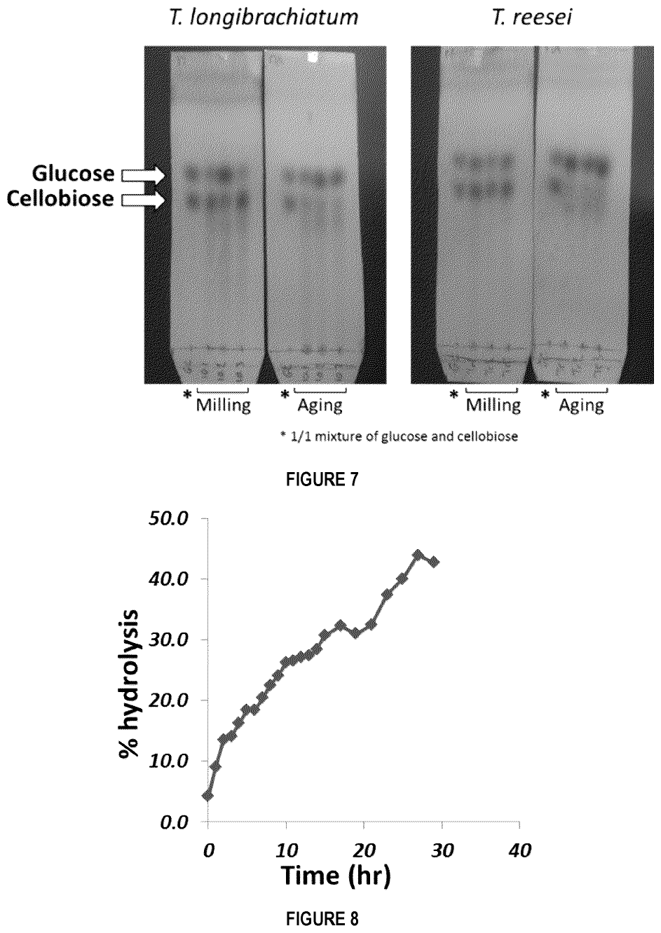

Analysis of the products revealed that while milling alone produced a mixture of glucose and cellobiose, if aging is carried out after milling the mixtures consisted mainly of glucose—see FIG. 7.

Preliminary studies to scale up the process (from 200 mg to 5 g) in a planetary mill (as opposed to a ball mill) for more than 12 cycles showed encouraging results—see FIG. 8.

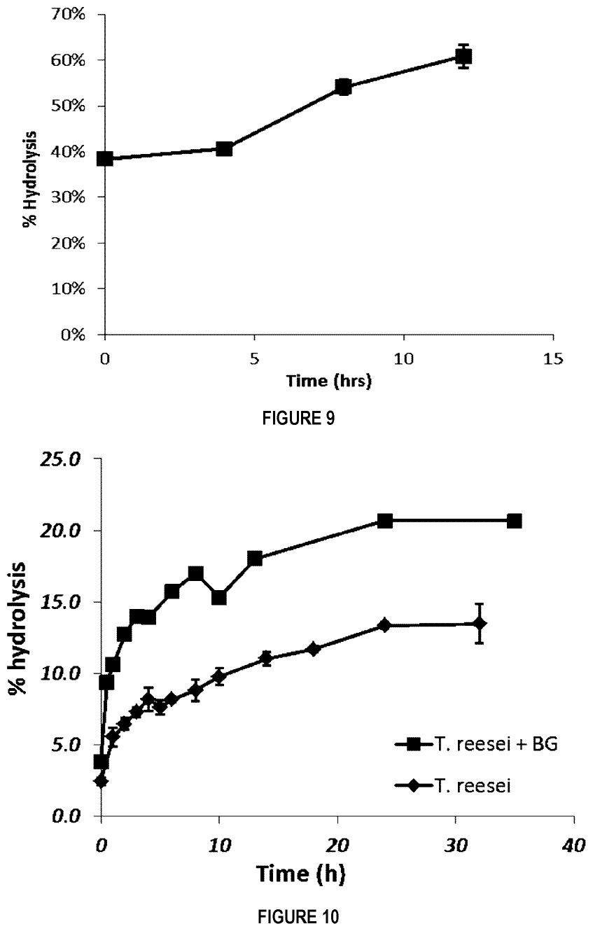

An attempt to recycle the enzyme and unreacted MCC after 12 (SMAA)n cycles was very encouraging: after an additional 12 (SMAA)n cycles carried out after recycling, an additional 20% conversion was observed, for a total of 60% under these conditions—see FIG. 9. The enzyme/unreacted MCC were separated from the aqueous product by centrifugation, and the resulting pellet was allowed to react further after addition of water to compensate for the removed water.

Experiments combining T. reesei cellulase (30 mg) with A. niger beta-glucosidase (BG) (30 mg) used to hydrolyze 2 g of MCC via one cycle of 5 min milling, followed by aging at 45° C. for various durations clearly demonstrated that the addition of beta-glucosidase significantly increased the yield of the process—see FIG. 10.

### EXAMPLE 13

### Hydrolysis of Chitin

These experiments reported in this Example were performed using commercial powdered chitin from shrimp shells and commercial lyophilized chitinase from Aspergillus niger (food grade, 208 U/g activity, 2% (w/w) protein content based on Bradford assay).

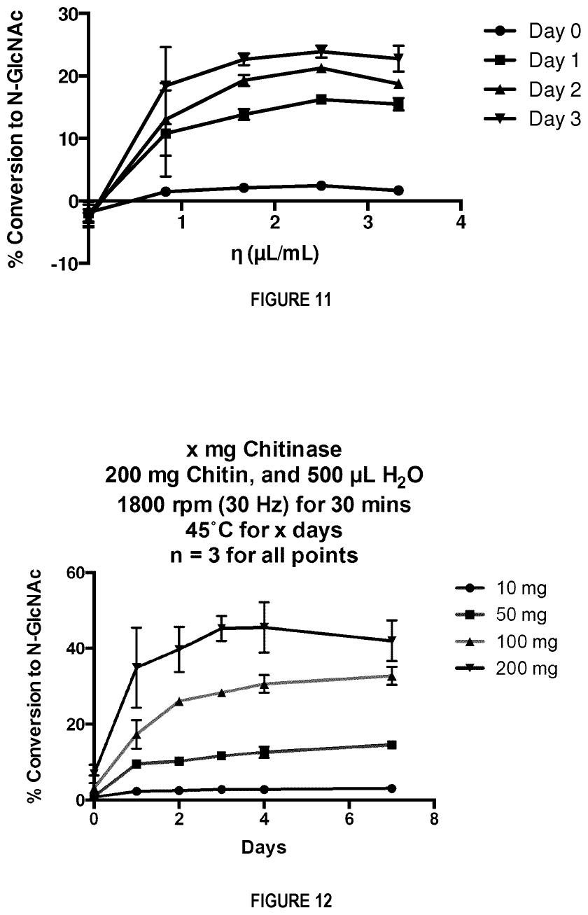

As a hydrolase, chitinase uses water as a substrate, and thus we have first optimized the amount of water needed. For an enzyme loading of 0.1% (w/w), the yield of the reaction during milling (30 Hz, 30 min, r.t.) was not significantly affected by the amount of water. When the samples were aged (45° C., 1-7 days) after milling, however, the yield was found to depend highly on the water content when η<1 μL/mg, but was more stable for η>1 μL/mg, and optimal at η˜1.6 μL/mg—see FIG. 11. Hence, unless otherwise noted, all experiments discussed below were performed with η=1.6 μL/mg.

The yield of the reaction was found to improve with increasing amount of enzyme, both after milling (30 Hz, 30 min, r.t.) and when milling was followed with aging (45° C., 1-7 days). FIG. 12 shows the percentage of chitin hydrolysis observed as a function of time for various enzyme loadings.

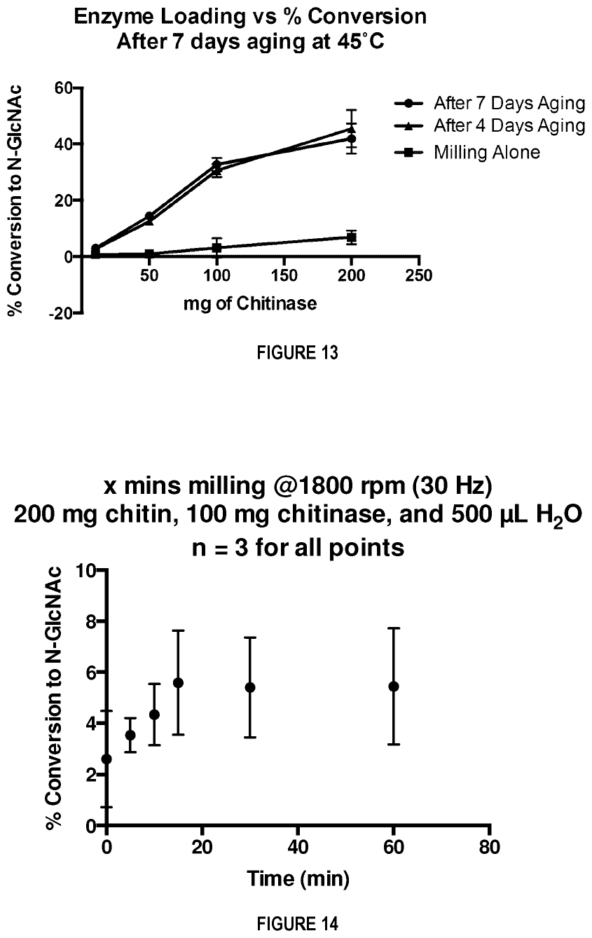

FIG. 13 shows the percentage of chitin hydrolysis observed as a function of enzyme loading when milling (30 Hz, 30 min, r.t.) alone and when milling followed by aging for 4 or 7 days at 45° C. We found that milling once followed by aging for 4 days gives a 30% conversion of chitin directly to N-acetylglucosamine (using 1% (w/w) protein/chitin).

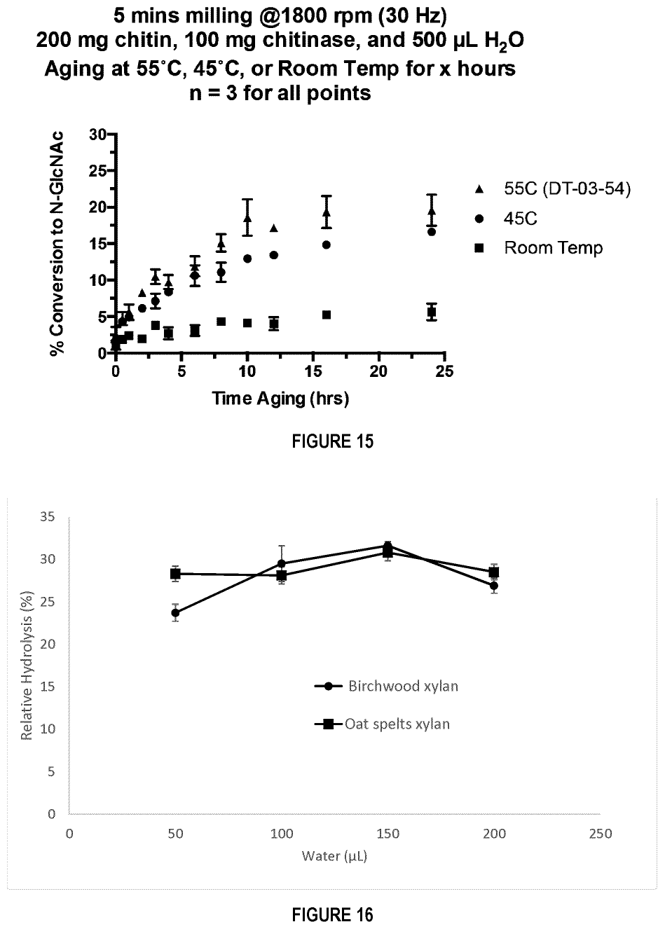

We looked at the kinetics of both the milling reaction (30 Hz, r.t.) and aging (45° C. and 55° C.) in order to identify the best conditions for successive SMAA reactions (SMAA)ₙ. The results, see FIGS. 14 and 15, suggest that such reactions should be optimal with ˜5-20 min of milling, followed by 5-10 hours of aging (both temperature give similar results). FIG. 14 shows the percentage of chitin hydrolysis observed as a function of milling time. FIG. 15 shows the percentage of chitin hydrolysis observed as a function of aging time (after milling for 5 mins at 30 Hz) at three temperatures (room temp, 45° C., and 55° C.).

A preliminary (SMAA)ₙ experiment involving 3 cycles of 15 min milling (30 Hz, r.t.) followed by 8 hours of aging at 45° C. (total of 1 day) with 1% (w/w) protein/chitin, gave ˜20% conversion (compared to ˜15% with one cycle of milling 30 min+aging 1 day).

### EXAMPLE 14

### Hydrolysis of Xylan

These studies were performed using commercial birchwood xylan and oat spelt xylan. We used Thermomyces lanuginosis xylanase produced in an engineered Aspergillus oryzae strain. The enzyme preparation was purchased as a lyophilized powder of 0.4% protein content (Bradford assay).

Preliminary results showed that xylanase can work well under milling conditions (30 Hz, 30 min) with an enzyme loading of 0.08% and η=1.6 μL/mg in the absence of bulk solvent.

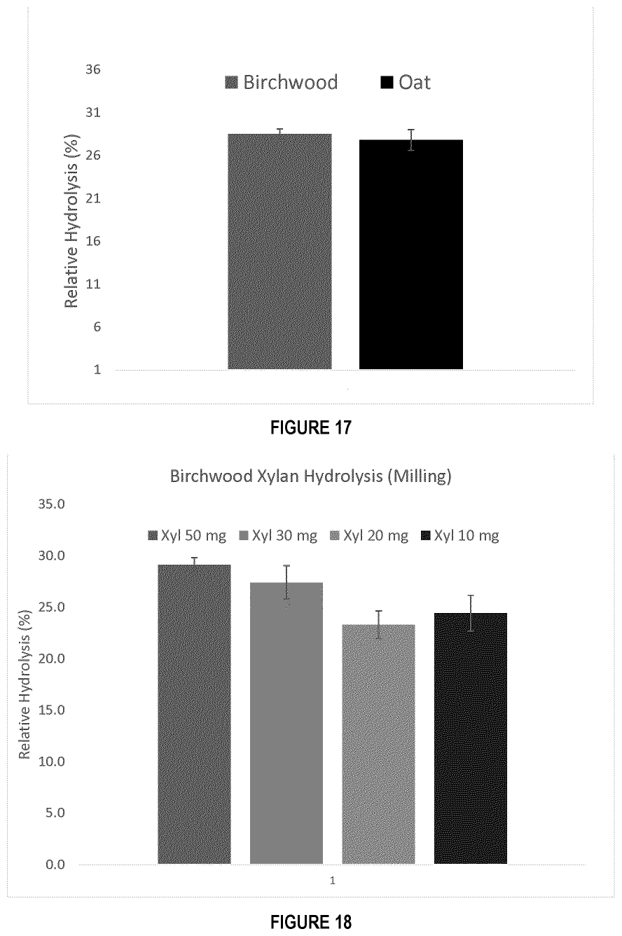

We optimized the amount of water in the reaction. Using an enzyme loading of 0.1% protein/xylan (w/w), we observed that xylan hydrolysis by xylanase under milling conditions (30 Hz, 30 min, r.t.) was only slightly affected by variations in η, with an optimum yield at η=0.6 μL/mg—see FIG. 16. Under these conditions we observed 30% conversion of xylan into xylose after 30 min of milling in the presence of 0.1% protein/xylan (w/w)—see FIG. 17.

Furthermore, still in the same conditions (enzyme loading varying from 0.1% to 0.5% protein/xylan (w/w), milling: 30 Hz, 30 min, r.t.), we found increased conversion as the amount of enzyme was increased, but the effect was relatively small—see FIG. 18.

### EXAMPLE 15

### Hydrolysis of Lignocellulosic Biomass

Experiments were performed on raw biomass obtained locally (cedar tree saw dust and hay), and on raw biomass of known cellulose and xylan content obtained from logen: sugarcane bagasse (“SB”, 40% cellulose and 22% xylan) and wheat straw (“WS”, 34% cellulose and 20% xylan).

Typically, the experiments consisted of pre-milling (or not) of the biomass, without any added water or enzyme, for 5 min (30 Hz, r.t.), followed by milling (5 min-1 h, 30 Hz, r.t.) in the presence of the reactant water and the desired enzyme, and finally allowing the sample to age for 1 h-7 days at room temperature (r.t.). Sometimes milling and aging are repeated over multiple cycles ((SMAA)ₙ also called RAging herein).

### Biomass Cellulose Degradation

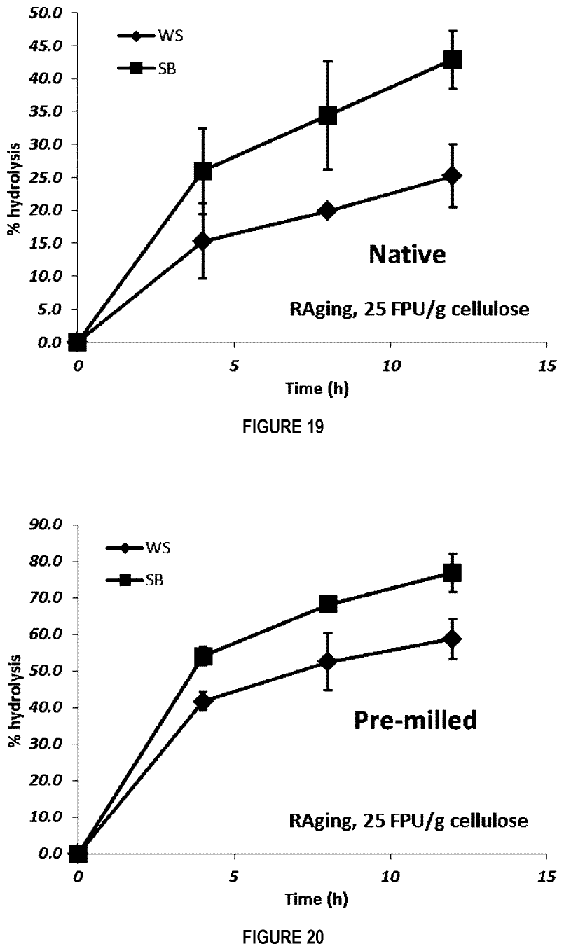

(SMAA)ₙ reactions of T. longibrachiatum cellulase (25 FPU/g) on sugarcane bagasse and wheat straw were found to proceed well, even on untreated (not pre-milled) raw biomass, with a better yield from sugarcane bagasse—see FIG. 19, η=1.3 μL/mg, 12 cyles of 5 min milling (30 Hz, r.t.) followed by 55 min aging at 55° C. N.B. the yields are based on the known cellulose content of each biomass sample.

In the same conditions, pre-milling of the biomass for 5 min before milling and aging, led to almost 80% conversion of sugarcane bagasse to glucose, and almost 60% conversion of wheat straw—see FIG. 20.

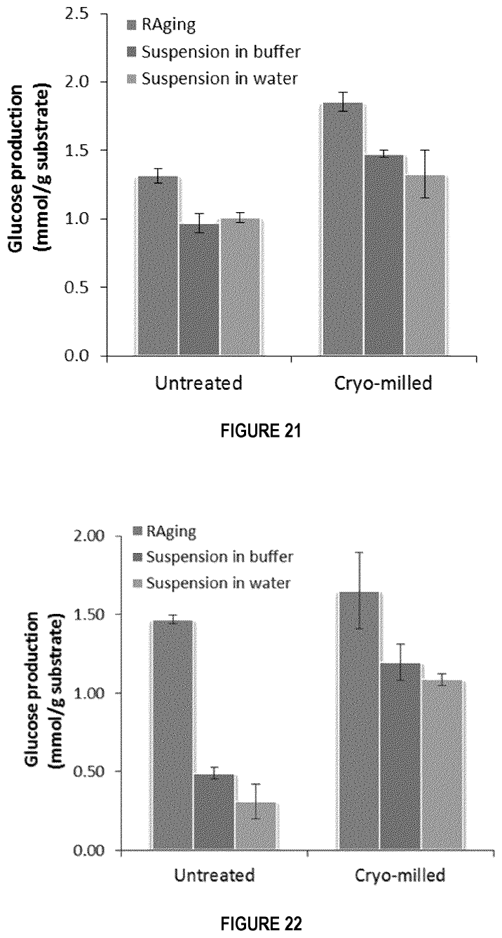

We compared the conversion of local samples of hay and cedar tree saw dust (unknown cellulose content) into glucose by cellulase using (SMAA)ₙ (also called RAging) in the above conditions versus standard slurry conditions—see FIGS. 21 and 22. NB. These standard slurry conditions are identified as “suspension in buffer” and “suspension in water” in FIGS. 21 and 22. The process of the invention was superior in all cases, with or without pre-milling (identified as cryo-milled in FIGS. 21 and 22).

### Biomass Xylan Degradation

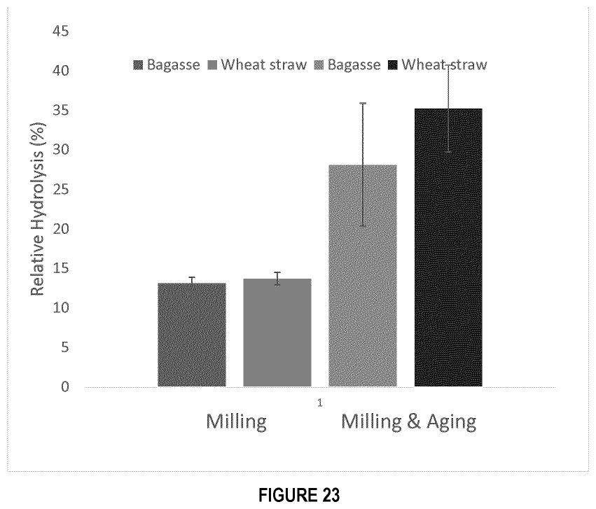

We studied the xylan degradation in sugarcane bagasse and wheat straw. Preliminary results show ˜13% hydrolysis of the xylans after 30 min of milling (30 Hz, r.t.) of either biomass using T. lanuginosus xylanase (0.1% w/w) at η=0.6 μL/mg. When the samples were further allowed to age at 55° C. for 3 days, the percent conversion raised to 30% for sugarcane bagasse and 35% for wheat straw, respectively—see FIG. 23.

This Example indicates that the process of the invention is less affected by the complex matrix found in raw biomass than the traditional aqueous process. Not having to chemically pre-treat the biomass is a significant advantage of our process.

The scope of the claims should not be limited by the preferred embodiments set forth in the examples above, but should be given the broadest interpretation consistent with the description as a whole.

### REFERENCES

The present description refers to a number of documents, the content of which is herein incorporated by reference in their entirety. These documents include, but are not limited to, the following:
US 2014/0024084;
US 2014/0147895;
US 2016/0002689;
US 2016/00032339;
U.S. Pat. No. 8,062,428
U.S. Pat. No. 8,647,468;
WO 2009/005390;
Bommarius, Biocatalysis: A Status Report, Annual Review of Chemical and Biomolecular Engineering, 2015, 6, pp. 319-345.
Klibanov, Improving enzymes by using them in organic solvents, Nature, 2001, 409, 241.
Mais et al., Enhancing the Enzymatic Hydrolysis of Cellulosic Materials Using Simultaneous Ball Miling, Applied Biochemistry and Biotechnology, Vols. 98-100, 2002, pp. 815-832;
Mais et al., Influence of Mixing Regime on Enzymatic Saccharification of Steam-Exploded Softwood Chips, Applied Biochemistry and Biotechnology, Vols. 98-100, 2002, pp. 463-472;
Olson et al., Recent Progress in Consolidated Bioprocession, Current Opinion in Biotechnology, 2012, 23, 396-405.
Rightmire and Hanusa, Advances in organometallic synthesis with mechanochemical methods, Dalton Trans., 2016, 455, 2352.
Suslick, Mechanochemistry and sonochemistry: concluding remarks, Faraday Discuss., 2014, 170, 411.
Ghose, T. K. 1987. “Measurement of Cellulase Activities.” Pure & Appl. Chem. 59: 257-268.

1. A method for the enzymatic saccharification of a polysaccharide, the method comprising:
a) the step of contacting the polysaccharide with a hydrolase and water, in the absence of solvent, thereby forming a solid reaction mixture; and
b) the step of:
b)-i. mixing and then incubating the solid reaction mixture,
b)-ii. milling the solid reaction mixture, or
b)-iii. milling and then incubating the solid reaction mixture.

2. (canceled)

3. (canceled)

4. (canceled)

5. The method of claim 1, wherein the water is in the form of pure water or in the form of an aqueous buffer, and wherein the solid reaction mixture has a ratio η of liquid volume, in μL, to total solid weight, in mg, between about 0.01 and about 3 μL/mg.

6. (canceled)

7. The method of claim 1, wherein the polysaccharide is provided in the form of lignocellulosic biomass.

8. The method of claim 7, wherein the lignocellulosic biomass is comminuted prior to step a).

9. The method of claim 7, wherein the hydrolase comprises one or more cellulase, one or more hemicellulase (preferably a xylanase), or a combination thereof, preferably a combination thereof.

10. (canceled)

11. (canceled)

12. (canceled)

13. (canceled)

14. (canceled)

15. The method of claim 1, wherein the polysaccharide comprises a cellulose

16. The method of claim 15, wherein the hydrolase comprise one or more cellulase and wherein the one or more cellulase exhibits two or more of the following types of activity: endocellulase activity, exocellulase activity, or β-glucosidase activity.

17. (canceled)

18. (canceled)

19. (canceled)

20. (canceled)

21. (canceled)

22. The method of claim 1, wherein the polysaccharide comprises a hemicellulose.

23. The method of any one of claim 22, wherein the hydrolase comprises a xylanase and wherein the xylanase is a xylanase from Thermomyces lanuginosis.

24. (canceled)

25. (canceled)

26. The method of claim 1, wherein the polysaccharide comprises chitin.

27. (canceled)

28. (canceled)

29. The method of claim 26, wherein the hydrolase comprises a chitinase and wherein the chitinase is a chitinase from Aspergillus niger, or S. griseus, or Amycoiaptosis orientalis.

30. (canceled)

31. (canceled)

32. (canceled)

33. (canceled)

34. (canceled)

35. The method of claim 1, wherein the solid reaction mixture comprises between about 1V and about 20V of water, V being the volume of the stoichiometric amount of water necessary to achieve a complete hydrolysis of the polysaccharide.

36. The method of claim 1, wherein the solid reaction mixture has a hydrolase concentration of about 0.01 w/w % to about 50 w/w %, based on the weight of the polysaccharide.

37. (canceled)

38. The method of claim 1, wherein in step a), the hydrolase is added to the polysaccharide in dry form.

39. (canceled)

40. (canceled)

41. (canceled)

42. The method of claim 1, wherein in step a), the hydrolase is added to the polysaccharide in the form of a solution of the hydrolase in the water.

43. (canceled)

44. (canceled)

45. (canceled)

46. (canceled)

47. (canceled)

48. (canceled)

49. (canceled)

50. (canceled)

51. (canceled)

52. (canceled)

53. The method of claim 1, wherein step b) comprises step b)-ii milling the solid reaction mixture.

54. The method of claim 1, wherein step b) comprises step b)-i mixing and then incubating the solid reaction mixture.

55. The method of claim 1, wherein step b) comprises step b)-iii milling and then incubating the solid reaction mixture.

56. (canceled)

57. The method of claim 55, comprising after step b)-iii, the step c′) of milling and then incubating the solid reaction mixture.

58. The method of claim 57, further comprising after step c′), the step of repeating step c′) one or more times.

59. (canceled)

60. (canceled)

61. (canceled)

62. (canceled)

63. (canceled)

64. (canceled)

65. (canceled)

66. (canceled)

67. (canceled)

68. (canceled)

69. (canceled)
